(19) United States
(12) Patent Application Publication
(10) Pub. No.: US 2009/0036472 A1
(43) Pub. Date: Feb. 5, 2009

(54) AZABICYCLO DERIVATIVES AS ANTI-INFLAMMATORY AGENTS

(76) Inventors: Venkata P. Palle, Gurgaon (IN); Ashwani Verma, New Delhi (IN); Rakesh Kumar Singh, Delhi (IN); Yogesh Bhaskarrao Waman, Nashik (IN); Arti Walia, Ambala (IN); Abhijit Ray, New Delhi (IN); Geeta Sharma, New Delhi (IN)

Correspondence Address: RANBAXY INC., 600 COLLEGE ROAD EAST, SUITE 2100, PRINCETON, NJ, 08540, US

(21) Appl. No.: 11/815,251

(22) PCT Filed: Feb. 1, 2006
(86) PCT No.: PCT/IB2006/000177

§ 371 Date: Mar. 25, 2008

(30) Foreign Application Priority Data
Feb. 2, 2005 (IN) 211DEL2005

(51) Int. Cl. C07D 487/04; A61K 31/519; A61P 29/00; A61P 19/02; A61P 11/00; A61P 9/00; A61P 3/00; A61P 3/10; A61P 1/00

(52) U.S. Cl. 514/264.11; 544/279

(57) ABSTRACT

The present invention relates to novel azabicyclo derivatives of Formula (I) as anti-inflammatory agents which are useful for inhibition and prevention of inflammation and associated pathologies including inflammatory and autoimmune diseases such as sepsis, rheumatoid arthritis, inflammatory bowel disease, type-1 diabetes, asthma, chronic obstructive pulmonary disorder, organ transplant rejection and psoriasis.

### FIELD OF THE INVENTION

The present invention relates to novel azabicyclo derivatives as anti-inflammatory agents.

The compounds of this invention were useful for inhibition and prevention of inflammation and associated pathologies including inflammatory and autoimmune diseases such as sepsis, rheumatoid arthritis, inflammatory bowel disease, type-1 diabetes, asthma, chronic obstructive pulmonary disorder, organ transplant rejection and psoriasis.

This invention also relates to pharmacological compositions containing the compounds of the present invention and the methods of treating sepsis, rheumatoid arthritis, inflammatory bowel disease, type-1 diabetes, asthma, chronic obstructive pulmonary disorder, organ transplant rejection and psoriasis, and other inflammatory and/or autoimmune disorders, using the compounds.

### BACKGROUND OF THE INVENTION

During the last decade, numerous studies have focused on the roles played by cytokines, a unique class of intercellular regulatory proteins, in the pathogenesis of many diseases. Cytokines play a crucial role in initiating, maintaining, and regulating immunological and inflammatory processes. Advances in our understanding of their role in immune and inflammatory disorders have led to the development of cytokine-based therapies—that is, therapies that aim to inhibit or restore the activity of specific cytokines. Today, drugs that block inflammatory cytokines, such as tumor necrosis factor-alpha (TNF-α), are among the most successful agents being introduced to the market.

Elevated levels of proinflammatory cytokines viz TNF-α and IL-β are associated with the pathogenesis of many immune mediated inflammatory disorders like sepsis, rheumatoid arthritis, inflammatory bowel disease, type-1 diabetes, asthma, chronic obstructive pulmonary disorder, organ transplant rejection and psoriasis. Inflammation is regulated by a large number of pro- and anti-inflammatory mediators, which include cytokines, eicosanoids, nitric oxide, and reactive oxygen species. The central role of these inflammatory mediators in the pathogenesis of both chronic and acute inflammatory diseases is well documented. Until a few years ago, inflammatory disorders were treated primarily with relatively non-selective anti-inflammatory agents, such as corticosteroids and various non-steroidal anti-inflammatory drugs. In recent years, novel therapies have been developed that specifically interfere with the action of selected pro-inflammatory mediators, such as TNF-α and PGE-2. These specific anti-inflammatory therapies have already proven to be very successful in the treatment of rheumatoid arthritis, inflammatory bowel disease, and several other inflammatory diseases.

The development of protein-based therapies that inhibit the activities of tumour-necrosis factor-α (TNF-α), including etanercept (Enbrel; Amgen/Wyeth), infliximab (Remicade; Centocor), and adalimumab (Humira; Abbott), has been an important advancement in the treatment of autoimmune diseases such as rheumatoid arthritis. The approval of Kineret—an interleukin-1 (IL-1) receptor antagonist—further indicates the clinical activity of protein-based therapies that regulate cytokine activities. However, current injectable therapies have associated limitations and risks, including the potential for increased malignancies and infections and increased congestive heart failure. Studies in rodent models have provided evidence that targeting specific pathways involved in TNF-α activities are effective approaches to interrupting the pro-inflammatory process. Oral small molecules that regulate these pathways should be the next significant advancement in the treatment of chronic inflammatory diseases when used either as a monotherapy or in combination with the current injectables.

Numerous studies have now clearly established that the pathogenesis of inflammatory diseases requires cytokine-mediated communication between endothelial cells, infiltrating leukocytes, resident macrophages, mast cells, epithelial cells and osteoclasts. The p38 mitogen activated protein kinase (p38MAPK) regulates cytokine levels and therefore plays a central role in both the cellular infiltration and activation responses associated with inflammatory diseases.

The p38 MAPK is a member of a large family of MAPK's whose signalling pathways also include the extracellular regulated kinases (ERK) & the c-jun N terminal kinases (JNK). MAP kinases are Serine Threonine Kinases that transduce environmental stimuli to the nucleus and they themselves are activated by upstream MAPK kinases by phosphorylation on both Tyrosine and Threonine residues. The MAPK pathways are involved in alterations in cell physiology resulting from a variety of stimuli and control cell death, cell cycle machinery, gene transcription and protein translation. p38α MAPK was first identified as a tyrosine phosphorylated protein in LPS (Lipopolysaccharide) stimulated macrophages. The human p38α MAPK was identified as the target of pyridinyl imidazole compounds (cytokine suppressive anti-inflammatory drugs) that were known to block TNF-α and IL-1 release from LPS stimulated monocytes. After the cloning of first p38 MAPK (p38α), additional members of the p38 MAPK family were cloned by homology, including the p38α, p38β and p38γ.

The p38 pathway controls the activity of multiple transcription factors and the expression of many genes. There is ample evidence implicating a pivotal role for p38 in inflammatory processes mediated by IL-1 and TNF-α. p38 inhibitors have been shown to effectively block both TNF-α and IL-1 biosynthesis by LPS stimulated human monocytes. In addition, p38 MAPK also plays a role in the production of IL-4, IL-6, IL-8 and IL-12. p38 MAPK is also critical for cell response to certain cytokines. Treatment of human neutrophils with GM-CSF, TNF-α or TGF-α results in p38 activation. GM-CSF and TNF-α are potent enhancers of neutrophil respiratory activity suggesting a role for p38 MAPK in respiratory burst.

p38 has also been implicated in the induction of cyclooxygenase-2 (COX-2) in LPS induced monocytes. COX-2 enzyme is the key enzyme in the production of prostaglandins from arachidonic acid. Inhibitors of p38 MAP kinase are also expected to inhibit COX-2 expression. Accordingly inhibitors of cytokine synthesis would be expected to be effective in disorders currently treated with NSAID's. These disorders include acute and chronic pain as well as symptoms of inflammation and cardiovascular disease.

Compounds, which modulate release of one or more of the aforementioned inflammatory cytokines, were useful in treating diseases associated with the release of these cytokines.

PCT Application WO 01/44258 discloses bone-targeting groups useful for treating a variety of disorders and conditions. PCT Application WO 02/18380 and U.S. Pat. Nos. 6,518,276 and 6,506,749 discloses 7-oxopyridopyrimidines as inhibitors of cell proliferation. PCT Application WO 03/057165 describes the compositions and methods for prevention and treatment of amyloid-O-peptide related disorders. U.S. Pat. No. 6,316,464 discloses compounds as p-38 kinase inhibitors. U.S. Pat. No. 6,451,804 discloses heteroalkylamino substituted bicyclic nitrogen heterocycles. U.S. Pat. No. 6,696,566 discloses 6-substituted pyrido-pyrimidines useful for the treatment of p-38 mediated disorders. U.S. Pat. No. 6,479,507 discloses p-38 kinase inhibitors. U.S. Publication No. 2003/0153586 discloses 7-oxo-pyridopyridopyrimidines for the treatment of p-38 mediated disorders. WO 2003/00270 discloses pyridopyrimidones and uses thereof. U.S. Pat. No. 6,630,485 discloses p-38 kinase inhibitors, pharmaceutical compositions containing them, method for their use, and methods for preparing these compounds. WO 04/019210 discloses pyridopyrimidine, naphthyridines and pyriodopyrazine derivatives as cyclin dependent kinase and tyrosine kinase inhibitors.

### SUMMARY OF THE INVENTION

The present invention provides novel azabicyclo derivatives, which were used for the inhibition and prevention of inflammation and associated pathologies such as sepsis, rheumatoid arthritis, inflammatory bowel disease, type-1 diabetes, asthma, chronic obstructive pulmonary disorder, organ transplant rejection and psoriasis.

Pharmaceutically acceptable salts, pharmaceutically acceptable solvates, enantiomers, diastereomers or N-oxides of these compounds having the same type of activity are also provided.

Pharmaceutical compositions containing the compounds, and which may also contain pharmaceutically acceptable carriers or diluents, which may be used for the treatment of inflammatory and autoimmune diseases such as sepsis, rheumatoid arthritis, inflammatory bowel disease, type-1 diabetes, asthma, chronic obstructive pulmonary disorder, organ transplant rejection and psoriasis.

Other aspects will be set forth in accompanying description which follows and in part will be apparent from the description or may be learnt by the practice of the invention.

In accordance with one aspect, there is provided a compound having the structure of Formula I

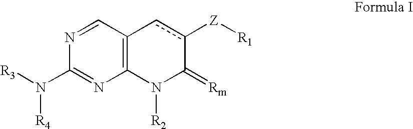

and its pharmaceutically acceptable salts, pharmaceutically acceptable solvates, esters, enantiomers diastereomers, N-oxides, polymorphs, metabolites;
wherein
R₁ is alkyl, alkenyl, alkynyl, cycloalkyl, aryl, aralkyl, heteroaryl, heterocyclyl, heteroarylalkyl, or heterocyclylalkyl;
when Rₘ is oxygen or sulphur
R₂ is hydrogen, alkyl, alkenyl, alkynyl, cycloalkyl, aryl, aralkyl, heteroaryl, heterocyclyl, heterocyclylalkyl or heteroarylalkyl;
when Rₘ is —NH, —N-acyl, —N(CN), —N(NO₂), —C(R₃)₂ or —CH(NO₂)
R₂ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y} or —NHC(═O)Rₓ; [symbol]
represents a single bond or a double bond;
R₃ is hydrogen, alkyl, alkenyl, alkynyl, cycloalkyl, aryl, heterocyclyl, heteroaryl, aralkyl, heteroarylalkyl or heterocyclylalkyl;
R₄ is

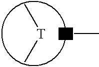

(wherein

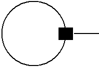

represents a cyclic ring containing 4-8 carbon atoms wherein 1-3 carbon atoms may optionally be replaced by heteroatoms selected from oxygen, —NH or sulphur; T is —(CH₂)ₙ—, —CH(Q)CH₂—, —CH₂CH(Q)CH₂—, —CH(Q)-, —CH₂—O—CH₂—, —CH₂—NH—CH₂—, —CH₂—N(CH₃)—CH₂);
n is an integer selected from 0-3 (wherein when n is zero then T represents a direct bond);
R₅ is alkyl, alkenyl, alkynyl, cycloalkyl, —NRₚR_{q} (wherein Rₚ and R_{q} are independently selected from the group consisting of hydrogen, alkyl, alkenyl, alkynyl, cycloalkyl, aryl, aralkyl, heterocyclyl, heteroaryl, heterocyclylalkyl or heteroarylalkyl; Rₚ and R_{q} may also together join to form a heterocyclyl ring), aryl, aralkyl, heteroaryl, heterocyclyl, heterocyclylalkyl or heteroarylalkyl;
R₆ is alkyl, alkenyl, alkynyl, cycloalkyl, aryl, aralkyl, heteroarylalkyl or heterocyclylalkyl;
Z is a direct bond, oxygen, sulphur, —NH or —(CH₂)ₙ;
   Rₓ and R_{y} are independently selected from the group consisting of hydrogen, alkyl, alkenyl, alkynyl, cycloalkyl, alkoxy, aryl, aralkyl, —SO₂R₅ (wherein R₅ is the same as defined above), heteroaryl, heterocyclyl, heteroarylalkyl or heterocyclylalkyl;
Q is alkyl, alkenyl, alkynyl, cycloalkyl, aryl, heteroaryl, heterocyclyl, aralkyl, heteroarylalkyl or heterocyclylalkyl.

In accordance with second aspect, there is provided a method for the treatment of mammal suffering from inflammation and associated pathologies.

In accordance with third aspect, there is provided a method for the treatment of mammal suffering from inflammatory diseases and associated pathologies including sepsis, rheumatoid arthritis, inflammatory bowel disease, type-1 diabetes, asthma, chronic obstructive pulmonary disorder, organ transplant rejection and psoriasis.

In accordance with fourth aspect, there are provided a pharmaceutical compositions containing the compounds, and which may also contain pharmaceutically acceptable carriers or diluents, which may be used for the treatment of inflammatory and autoimmune diseases such as sepsis, rheumatoid arthritis, inflammatory bowel disease, type-1 diabetes, asthma, chronic obstructive pulmonary disorder, organ transplant rejection and psoriasis.

In accordance with fifth aspect, there is provided a process for the preparation of compounds disclosed herein.

In accordance with sixth aspect, the compounds of the present invention are screened as p38 kinase inhibitors.

The following definitions apply to terms as used herein:

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NRₐ—, wherein Rₐ can be hydrogen, alkyl, alkenyl, alkynyl cycloalkyl or aryl. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further (referred herein as “substituted alkyl”) with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)Rₚ, —NRₚR_{q}, —C(═O)NRₚR_{q}, —NHC(═O)NRₚR_{q}, —C(═O)heteroaryl, C(═O)heterocyclyl, —O—C(═O)NRₚR_{q}{wherein Rₚ and R_{q} are independently selected from hydrogen, alkyl, alkenyl, alkynyl, cycloalkyl, alkoxy, cycloalkenyl, aryl, aralkyl, heterocyclyl, heteroaryl, heterocyclylalkyl, heteroarylalkyl}, nitro, hydroxyamino, alkoxyamino or S(O)ₘR₆₆ (wherein m is an integer from 0-2 and R₆₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NRₚR_{q}, —C(═O)NRₚR_{q}, —OC(═O)NRₚR_{q}, —NHC(═O)NRₚR_{q}(wherein Rₚ and R_{q} are the same as defined earlier), hydroxy, alkoxy, halogen, CF₃, cyano, and S(O)ₘR₆₆ (wherein m is an integer from 0-2 and R₆₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)ORₚ (wherein Rₚ is the same as defined earlier), S(O)ₘR₆₆ (wherein m is an integer from 0-2 and R₆₆ is as defined earlier), or —C(═O)NRₚR_{q}(wherein Rₚ and R_{q} are as defined earlier)}. Unless otherwise constrained by the definition, all substituents may be substituted further by 1-3 substituents selected from alkyl, carboxy, carboxyalkyl, —NRₚR_{q}, —C(═O)NRₚR_{q}, —O—C(═O)NRₚR_{q} (wherein Rₚ and R_{q} are the same as defined earlier) hydroxy, alkoxy, halogen, CF₃, cyano, and S(O)ₘR₆₆ (wherein m is an integer from 0-2 and R₆₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. It can be optionally interrupted by atom(s) or group(s) independently chosen from oxygen, sulfur, phenylene, sulphinyl, sulphonyl and —NRₐ—, wherein Rₐ can be hydrogen, alkyl, alkenyl, alkynyl, cycloalkyl or aryl. In the event that alkenyl is attached to a heteroatom, the double bond cannot be alpha to the heteroatom. Alkenyl groups may be substituted further (referred to herein as “substituted alkenyl”) with one or more substituents selected from alkyl, alkenyl, alkynyl, alkoxy, cycloalkyl, cycloalkenyl, acyl, acylamino, acyloxy, —NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro, or SO₂R₆₆ (wherein R₆₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NRₚR_{q}, —C(═O)NRₚR_{q}, —O—C(═O)NRₚR_{q} (wherein Rₚ and R_{q} are the same as defined earlier) and —SO₂R₆₆ (wherein R₆₆ is same as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. It can be optionally interrupted by atom(s) or group(s) independently chosen from oxygen, sulfur, phenylene, sulphinyl, sulphonyl and —NRₐ—, wherein Rₐ can be hydrogen, alkyl, alkenyl, alkynyl, cycloalkyl or aryl. In the event that alkynyl is attached to a heteroatom, the triple bond cannot be alpha to the heteroatom. Alkynyl groups may be substituted further (referred to herein as “substituted alkynyl”) with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)Rₚ, —NRₚR_{q}, —NHC(═O)NRₚR_{q}, —C(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NRₚR_{q}, —C(═O)NRₚR_{q}, —NHC(═O)NRₚR_{q}, —C(═O)NRₚR_{q} (wherein Rₚ and R_{q} are the same as defined earlier), cyano, or S(O)ₘR₆₆ (wherein m is an integer from 0-2 and R₆₆ is same as defined earlier). Groups such as ethynyl, (—C≡H), propargyl (or propynyl, —CH₂C≡CH), and the like exemplify this term.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo [2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NRₚ, —NHC(═O)NRₚR_{q}, —NHC(═O)Rₚ, —C(═O)NRₚR_{q}, —O—C(═O)NRₚ (wherein Rₚ and R_{q} are the same as defined earlier), nitro, heterocyclyl, heteroaryl, heterocyclylalkyl, heteroarylalkyl, or S(O)ₘR₆₆ (wherein m is an integer from 0-2 and R₆₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NRₚR_{q}, —C(═O)NRₚRₐ, —NHC(═O)NRₚR_{q}, —C(═O)NRₚR_{q} (wherein Rₚ and R_{q} are the same as defined earlier), cyano or S(O)ₘR₆₆ (wherein m is an integer from 0-2 and R₆₆ is same as defined earlier).

The term “alkoxy” denotes the group O-alkyl wherein alkyl is the same as defined above.

The term “aryl” herein refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₛ (wherein Rₛ is hydrogen, alkyl, alkenyl, alkynyl, cycloalkyl, aralkyl, heterocyclylalkyl, heteroarylalkyl), NHC(═O)Rₚ, —NRₚR_{q}, —C(═O)NRₚR_{q}, —NHC(═O)NRₚR_{q}, —O—C(═O)NRₚR_{q}, S(O)ₘR₆₆ (wherein m is an integer from 0-2 and R₆₆ is same as defined earlier), carboxy, optionally substituted heterocyclyl, heteroaryl, heterocyclylalkyl, heteroarylalkyl, amino carbonyl amino, mercapto, haloalkyl, optionally substituted aryl, optionally substituted heterocyclylalkyl, thioalkyl, —CONHRₚ, —OCORₚ, —CORₚ, —NHSO₂Rₚ, or —SO₂NHRₚ (wherein Rₚ and R_{q} are the same as defined earlier). The aryl group optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “carboxy” as defined herein refers to —C(═O)OH.

The term “aryloxy” denotes the group O-aryl, wherein aryl is as defined above.

The term “heteroaryl,” unless otherwise specified, refers to an aromatic ring structure containing 5 or 6 ring atoms, or a bicyclic or tricyclic aromatic group having from 8 to 14 ring atoms, with one or more heteroatom(s) independently selected from N, O or S. Heteroaryl groups can be optionally substituted with 1 to 4 substituent(s) (referred herein as “substituted heteroaryl”) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NRₚR_{q}, CH═NOH, —(CH₂)_{w}C(═O)Rₜ {wherein w is an integer from 0-4 and Rₜ is hydrogen, hydroxy, ORₚ, NRₚR_{q}, —NHOR_{z}, or —NHOH}, —C(═O)NRₚR_{q} and —NHC(═O)NRₚR_{q}, S(O)ₘR₆₆, —O—C(═O)NRₚR_{q}, —O—C(═O)Rₚ, —O—C(═O)ORₚ (wherein m, R₆₆, Rₚ and R_{q} are as defined earlier, and R_{z} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, benzthiazinyl, benzthiazinonyl, benzoxazinyl, benzoxazinonyl, quinazonyl, carbazolyl phenothiazinyl, phenoxazinyl and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)Rₚ, —O—C(═O)ORₚ, —C(═O)NRₚR_{q}, S(O)ₘR₆₆, —O—C(═O)NRₚR_{q}, —NHC(═O)NRₚR_{q}, —NRₚR_{q}, NRₚR_{q}, mercapto, haloalkyl, thioalkyl, —COORₚ, —COONH Rₚ, —CO Rₚ, —NHSO₂ Rₚ, SO₂NH Rₚ (wherein m, R₆₆, Rₚ and R_{q} are as defined earlier) or guanidine. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine, and the like.

Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

“Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Thiocarbonyl” refers to —C(═S)H.

“Substituted thiocarbonyl” refers to —C(═S)R″, wherein R″ is selected from alkyl, cycloalkyl, aryl, aralkyl, heteroaryl, heterocyclyl, heteroarylalkyl or heterocyclylalkyl, amine or substituted amine.

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that were modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

### DETAILED DESCRIPTION OF THE INVENTION

The compounds of the present invention may be prepared by techniques well known in the art and familiar to a practitioner skilled in art of this invention. In addition, the compounds of the present invention may be prepared by, or example, processes described herein, although such processes are not the only means by which the compounds described may be synthesised. Further, the various synthetic steps described herein may be performed in an alternate sequence in order to give the desired compounds.

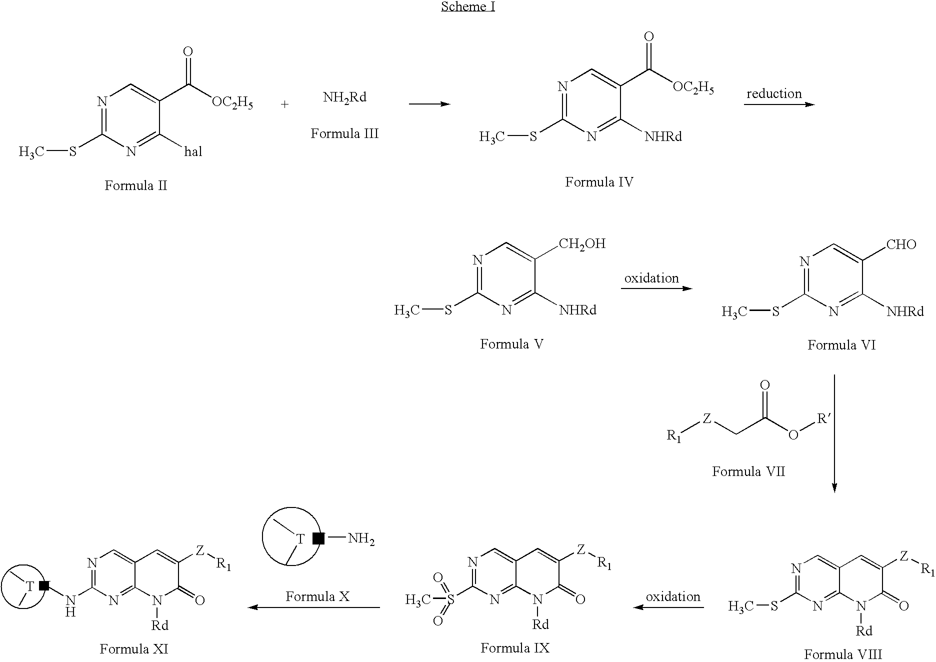

The compounds of Formulae XI and XII can be and were prepared by following the reaction sequence of Scheme I. Thus, a compound of Formula II [wherein hal is halogen (Cl, Br or I)] can be reacted with a compound of Formula III (wherein R_{d} is hydrogen, optionally substituted alkyl, cycloalkyl, aralkyl or aryl) to give a compound of Formula IV, which can undergo reduction to give a compound of Formula V, which can be further oxidized to give a compound of Formula VI, which can be reacted with an ester of Formula VII (wherein R′ is alkyl; R₁ and Z are the same as defined earlier) to give a compound of Formula VIII, which can be oxidized to give a compound of Formula IX, which can be reacted with a compound of Formula X [wherein T is the same as defined earlier], to give a compound of Formula XI.

The reaction of a compound of Formula II with a compound of Formula III to give a compound of Formula IV can be carried out in an organic solvent, for example tetrahydrofuran, dimethylformamide, dioxane or diethyl ether, in the presence of a base, for example, triethylamine, N-ethyldiisopropylamine, N-methylmorpholine or pyridine.

The compound of Formula IV can be reduced to give a compound of Formula V in an organic solvent, for example, tetrahydrofuran, dimethylformamide, dioxane or diethylether, with reducing agents, for example, lithium aluminium hydride, lithium borohydride, sodium cyanoborohydride or sodium borohydride.

The oxidation of a compound of Formula V to give a compound of Formula VI can be carried out in an organic solvent, for example, dichloromethane, dichloroethane, carbon tetrachloride or chloroform with an oxidizing agent for example, manganese dioxide, potassium permanganate, Dess-Martin periodinane (DMP), pyridinium dichromate (PDC), pyridinium chlorochromate (PCC) or chromic anhydride, although numerous other methods were employed (see, for example, Advanced Organic Chemistry, 4^{th} Edn., Merck, John Wiley & Sons, 1992).

The reaction of a compound of Formula VI with a compound of Formula VII to give a compound of Formula VIII can be carried out in an organic solvent, for example, N-methylpyrrolidinone, dimethylformamide, tetrahydrofuran, diethylether or dioxane in the presence of a base, for example, potassium carbonate, sodium carbonate or lithium carbonate, potassium bicarbonate, lithium bicarbonate or sodium bicarbonate.

The oxidation of a compound of Formula VIII to give a compound of Formula IX can be carried out with an oxidizing agent, for example, m-chloroperbenzoic acid or oxone (KHSO₅) in an organic solvent, for example, chloroform, carbon tetrachloride, dichloromethane, ethanol or tetrahydrofuran.

The reaction of a compound of Formula IX with a compound of Formula X to give a compound of Formula XI can be carried in the presence of a base, for example, pyridine, N-methylmorpholine, N-ethyldiisopropylamine, sodium hydride or triethylamine.

Alternatively, in some cases rather than using a compound of Formula IX, a compound of Formula VIII can be reacted directly with a compound of Formula X to give a compound of Formula XI.

Particular compounds are mentioned below:
6-(2-Chlorophenyl)-2-(3-azabicyclo[3.3.0]oct-3-yl-amino)-8-methylpyrido[2,3-d]pyrimidin-7(8H)-one (Compound No. 1),
6-(2-Methylphenyl)-2-(3-azabicyclo[3.3.0]oct-3-yl-amino)-8-methylpyrido[2,3-d]pyrimidin-7(8H)-one (Compound No. 2),
6-(2-Fluorophenyl)-2-(3-azabicyclo[3.3.0]oct-3-yl-amino)-8-methylpyrido[2,3-d]pyrimidin-7(8H)-one (Compound No. 3),
6-(2,4-Difluorophenyl)-2-(3-azabicyclo[3.3.0]oct-3-yl-amino)-8-methylpyrido[2,3-d]pyrimidin-7(8H)-one (Compound No. 4),
6-(3-Chlorophenyl)-2-(3-azabicyclo[3.3.0]oct-3-yl-amino)-8-methylpyrido[2,3-d]pyrimidin-7(8H)-one (Compound No. 5),
6-(4-Chlorophenyl)-2-(3-azabicyclo[3.3.0]oct-3-yl-amino)-8-methylpyrido[2,3-d]pyrimidin-7(8H)-one (Compound No. 6),
6-(4-Methylphenyl)-2-(3-azabicyclo[3.3.0]oct-3-yl-amino)-8-methylpyrido[2,3-d]pyrimidin-7(8H)-one (Compound No. 7),
6-(4-Fluorophenyl)-2-(3-azabicyclo[3.3.0]oct-3-yl-amino)-8-methylpyrido[2,3-d]pyrimidin-7(8H)-one (Compound No. 8),
6-(2-Methylphenyl)-2-(3-azabicyclo[3.3.0]oct-3-yl-amino)-8-benzylpyrido[2,3-d]pyrimidin-7(8H)-one (Compound No. 9),
6-(2-Methylphenyl)-2-(3-azabicyclo[3.3.0]oct-3-yl-amino)-8-(4-fluorobenzyl)-pyrido[2,3-d]pyrimidin-7(8H)-one (Compound No. 10),
6-(2-Methylphenyl)-2-(3-azabicyclo[3.3.0]oct-3-yl-amino)-8-(4-tert-butylbenzyl)-pyrido[2,3-d]pyrimidin-7(8H)-one (Compound No. 11),
6-(2-Methylphenyl)-2-(3-azabicyclo[3.3.0]oct-3-yl-amino)-8-(3-trifluoromethoxybenzyl)-pyrido[2,3-d]pyrimidin-7(8H)-one (Compound No. 12),
6-(2-Fluorophenyl)-2-(3-azabicyclo[3.3.0]oct-3-yl-amino)-8-(4-fluorobenzyl)-pyrido[2,3-d]pyrimidin-7(8H)-one (Compound No. 13),
6-(2-Fluorophenyl)-2-(3-azabicyclo[3.3.0]oct-3-yl-amino)-8-(3-chloro-2-fluorobenzyl)-pyrido[2,3-d]pyrimidin-7(8H)-one (Compound No. 14),
6-(2-Chlorophenyl)-2-(3-azabicyclo[3.3.0]oct-3-yl-amino)-8-benzylpyrido[2,3-d]pyrimidin-7(8H)-one (Compound No. 15),
6-(2-Chlorophenyl)-2-(3-azabicyclo [3.3.0]oct-3-yl-amino)-8-(4-fluorobenzyl)-pyrido[2,3-d]pyrimidin-7(8H)-one (Compound No. 16),
6-(2-Chlorophenyl)-2-(3-azabicyclo[3.3.0]oct-3-yl-amino)-8-(4-tert-butylbenzyl)-pyrido[2,3-d]pyrimidin-7(8H)-one (Compound No. 17),
6-(2-Chlorophenyl)-2-(3-azabicyclo[3.3.0]oct-3-yl-amino)-8-(3-trifluoromethoxybenzyl)-pyrido[2,3-d]pyrimidin-7(8H)-one (Compound No. 18),
6-(2-Methylphenyl)-2-(3-azabicyclo[3.3.0]oct-3-yl-amino)-pyrido[2,3-d]pyrimidin-7(8H)-one (Compound No. 30),
6-(2-Fluorophenyl)-2-(3-azabicyclo[3.3.0]oct-3-yl-amino)pyrido[2,3-d]pyrimidin-7(8H)-one (Compound No. 31),
6-(2-Methylphenyl)-2-(7-azabicyclo[4.3.0]non-7-yl-amino)-8-(cyclopropyl)-pyrido[2,3-d]pyrimidin-7(8H)-one (Compound No. 41),
6-(2-Methylphenyl)-2-(7-azabicyclo[4.3.0]non-7-yl-amino)-8-methyl-pyrido[2,3-d]pyrimidin-7(8H)-one (Compound No. 42),
6-(2-Chlorophenyl)-2-(7-azabicyclo[4.3.0]non-7-yl-amino)-8-methyl-pyrido[2,3-d]pyrimidin-7(8H)-one (Compound No. 43),
6-(2-Chlorophenyl)-2-(3-azabicyclo[3.3.0]oct-3-yl-amino)-8-cyclopropyl-pyrido[2,3-d]pyrimidin-7(8H)-one (Compound No. 52),
6-(2-Chlorophenyl)-2-(3-azabicyclo[3.3.0]oct-3-yl-amino)-7-oxopyrido[2,3-d]pyrimidin-8(7H)-yl acetonitrile (Compound No. 53),
6-(1-Naphthyl)-2-(3-azabicyclo[3.3.0]oct-3-yl-amino)-8-cyclopropyl-pyrido[2,3-d]pyrimidin-7(8H)-one (Compound No. 54),
6-(2-Chlorophenoxy)-2-(3-azabicyclo[3.3.0]oct-3-yl-amino)-8-cyclopropyl-pyrido[2,3-d]pyrimidin-7(8H)-one (Compound No. 55),
6-(3-Methylphenyl)-2-(3-azabicyclo[3.3.0]oct-3-yl-amino)-8-methyl-pyrido[2,3-d]pyrimidin-7(8H)-one (Compound No. 56),
6-(2-Methylphenoxy)-2-(3-azabicyclo[3.3.0]oct-3-yl-amino)-8-cyclopropyl-pyrido[2,3-d]pyrimidin-7(8H)-one (Compound No. 57),
6-(2-Methylphenoxy)-2-(3-azabicyclo[3.3.0]oct-3-yl-amino)-8-methylpyrido[2,3-d]pyrimidin-7(8H)-one (Compound No. 58),
6-(2-Fluorophenoxy)-2-(3-azabicyclo[3.3.0]oct-3-yl-amino)-8-cyclopropyl-pyrido[2,3-d]pyrimidin-7(8H)-one (Compound No. 59),
6-(4-Chlorophenoxy)-2-(3-azabicyclo[3.3.0]oct-3-yl-amino)-8-cyclopropylpyrido[2,3-d]pyrimidin-7(8H)-one (Compound No. 60),
6-(4-Chlorophenoxy)-2-(3-azabicyclo[3.3.0]oct-3-yl-amino)-8-methylpyrido[2,3-d]pyrimidin-7(8H)-one (Compound No. 61),
6-Methoxy-2-(3-azabicyclo[3.3.0]oct-3-yl-amino)-8-methylpyrido[2,3-d]pyrimidin-7(8H)-one (Compound No. 62),
6-(4-Isopropylphenyl)-2-(3-azabicyclo[3.3.0]oct-3-yl-amino)-8-methylpyrido[2,3-d]pyrimidin-7(8H)-one (Compound No. 63),
6-(2-Fluorophenyl)-2-(3-azabicyclo[3.3.0]oct-3-yl-amino)-8-(cyclopropyl)pyrido[2,3-d]pyrimidin-7(8H)-one (Compound No. 64),
6-(2,4-Dichlorophenyl)-2-(3-azabicyclo[3.3.0]oct-3-yl-amino)-8-methylpyrido[2,3-d]pyrimidin-7(8H)-one (Compound No. 65),
6-(4-Nitrophenyl)-2-(3-azabicyclo[3.3.0]oct-3-yl-amino)-8-methyl-pyrido[2,3-d]pyrimidin-7(8H)-one (Compound No. 66),
6-(3-Methoxyphenyl)-2-(3-azabicyclo[3.3.0]oct-3-yl-amino)-8-methylpyrido[2,3-d]pyrimidin-7(8H)-one (Compound No. 67),
6-(2,6-Dichlorophenyl)-2-(3-azabicyclo[3.3.0]oct-3-yl-amino)-8-methylpyrido[2,3-d]pyrimidin-7(8H)-one (Compound No. 68),
6-(2-Nitrophenyl)-2-(3-azabicyclo [3.3.0]oct-3-yl-amino)-8-methyl-pyrido[2,3-d]pyrimidin-7(8H)-one (Compound No. 69),
6-(3-Fluorophenyl)-2-(3-azabicyclo[3.3.0]oct-3-yl-amino)-8-methylpyrido[2,3-d]pyrimidin-7(8H)-one (Compound No. 70),
6-(2-Chloro-4-fluorophenyl)-2-(3-azabicyclo [3.3.0]oct-3-yl-amino)-8-methylpyrido[2,3-d]pyrimidin-7(8H)-one (Compound No. 71),
6-(4-Nitrophenoxy)-2-(3-azabicyclo[3.3.0]oct-3-yl-amino)-8-methyl-pyrido[2,3-d]pyrimidin-7(8H)-one (Compound No. 72),
6-(2-Chlorophenoxy)-2-(3-azabicyclo[3.3.0]oct-3-yl-amino)-8-methylpyrido[2,3-d]pyrimidin-7(8H)-one (Compound No. 73),
6-(2-Fluorophenoxy)-2-(3-azabicyclo[3.3.0]oct-3-yl-amino)-8-methylpyrido[2,3-d]pyrimidin-7(8H)-one (Compound No. 74),
6-(4-Chloro-2-fluorophenoxy)-2-(3-azabicyclo[3.3.0]oct-3-yl-amino)-8-methylpyrido[2,3-d]pyrimidin-7(8H)-one (Compound No. 75),
6-(4-Chloro-2-fluorophenoxy)-2-(3-azabicyclo[3.3.0]oct-3-yl-amino)-8-cyclopropyl-pyrido[2,3-d]pyrimidin-7(8H)-one (Compound No. 76),
6-(3-Methoxyphenoxy)-2-(3-azabicyclo[3.3.0]oct-3-yl-amino)-8-(cyclopropyl) -pyrido[2,3-d]pyrimidin-7(8H)-one (Compound No. 77),
6-(3-Methylphenoxy)-2-(3-azabicyclo[3.3.0]oct-3-yl-amino)-8-(cyclopropyl)-pyrido[2,3-d]pyrimidin-7(8H)-one (Compound No. 78).

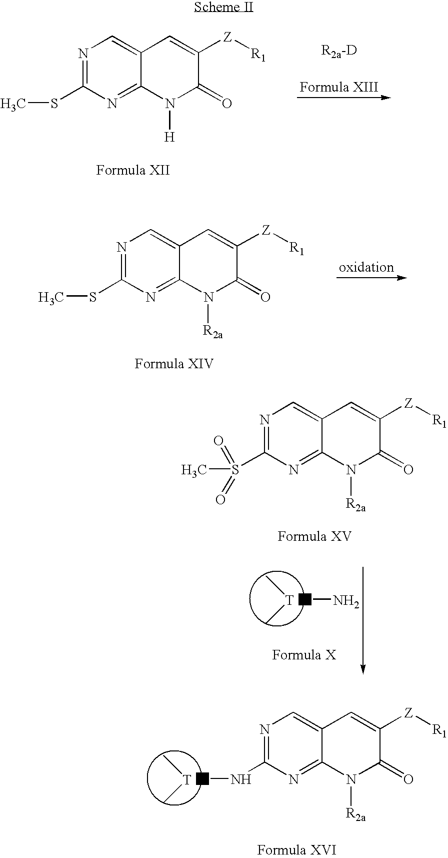

Compounds of Formula XVI can be and were prepared following the procedure as depicted in Scheme II. An exemplary reaction can comprise reacting a compound of Formula XII (wherein Z and R₁ are the same as defined earlier) with a compound of Formula XIII (wherein R₂ₐ is alkyl, alkenyl, alkynyl, cycloalkyl, aryl, aralkyl, heteroaryl, heterocyclyl, heterocyclylalkyl or heteroarylalkyl and D is hal (Br, Cl or I) or —OH) are the same as defined earlier) to give a compound of Formula XIV, which can undergo oxidation to give a compound of Formula XV, which can be reacted with a compound of Formula X (wherein T is the same as defined earlier) to give a compound of Formula XVI.

The reaction of a compound of Formula XII with a compound of Formula XIII (when D is hal) to give a compound of Formula XIV can be carried out in the presence of a base, for example, sodium hydride, potassium hydride or lithium hydride, in an organic solvent, for example, N-methylpyrrolidone, dimethylformamide, tetrahydrofuran or dimethylsulphoxide.

Alternatively, the reaction of a compound of Formula XII with a compound of Formula XIII (when D is hal) can be also carried out in the presence of a base, for example, potassium carbonate, sodium carbonate, lithium carbonate or sodium bicarbonate, and a catalyst, for example, tetrabutylammonium bromide, tetrabutylammonium iodide or tetrabutylammonium chloride.

The reaction of a compound of Formula XII with a compound of Formula XIII (when D is —OH) to give a compound of Formula XIV can be carried out in an organic solvent, for example, tetrahydrofuran, diethylether, dioxane, toluene, benzene or dimethylformamide, in the presence of a redox couple. The redox couple agents may be any one of those known to a person skilled in the art of organic synthesis. The oxidizing part of the redox couple can be, for example, diisopropylazodicarboxylate (DIAD), diethylazodicarboxylate (DEAD), N,N,N′N′-tetramethylazodicarboxamide (TMAD), 1,1′-(azodicarbonyl)dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′N′-tetraisopropylazodicarboxamide (TIPA). The reduction part of the redox couple can be, for example, a phosphine, for example, trialkylphosphine (such as tributylphosphine), triarylphosphine (such as triphenylphosphine), tricycloalkylphosphine (such as tricyclohexylphosphine) or triheteroarylphosphine. Phosphine reagents, together with a combination of aryl, alkyl or heteroaryl substituents, may also be used (such as diphenylpyridylphosphine).

The oxidation of a compound of Formula XIV to give a compound of Formula XV can be carried out with an oxidizing agent, for example, m-chloroperbenzoic acid or oxone (KHSO₅), in an organic solvent, for example, chloroform, dichloroethane, carbon tetrachloride, dichloromethane, ethanol or tetrahydrofuran.

The reaction of a compound of Formula XV with a compound of Formula X to give a compound of Formula XVI can be carried in the presence of a base, for example, pyridine, N-methylmorpholine, diisopropylethylamine, sodium hydride or triethylamine.

Alternatively, in some cases, rather than using a compound of Formula XV, a compound of Formula XIV can be reacted directly with a compound of Formula X to give a compound of Formula XVI.

Particular compounds are mentioned below:
6-(2-Methylphenyl)-2-(3-azabicyclo[3.3.0]oct-3-yl-amino)-8-(cyclopropyl methyl)pyrido[2,3-d]pyrimidin-7(8H)-one (Compound No. 19),
Ethyl 2-[6-(2-fluorophenyl)-2-(3-azabicyclo[3.3.0]oct-3-yl-amino)-7-oxopyrido[2,3-d]pyrimidin-8(7H)-yl]propanoate (Compound No. 20),
Ethyl 2-[6-(2-methylphenyl)-2-(3-azabicyclo [3.3.0]oct-3-yl-amino)-7-oxopyrido[2,3-d]pyrimidin-8(7H)-yl]propanoate (Compound No. 21),
6-(2-Fluorophenyl)-2-(3-azabicyclo[3.3.0]oct-3-yl-amino)-8-(cyclopentyl)pyrido[2,3-d]pyrimidin-7(8H)-one (Compound No. 22),
6-(2-Fluorophenyl)-2-(3-azabicyclo[3.3.0]oct-3-yl-amino)-8-(cyclopropyl methyl)-pyrido[2,3-d]pyrimidin-7(8H)-one (Compound No. 23),
6-(2-Methylphenyl)-2-(3-azabicyclo[3.3.0]oct-3-yl-amino)-8-(2-chloroethyl)-pyrido[2,3-d]pyrimidin-7(8H)-one (Compound No. 24),
6-(2-Chlorophenyl)-2-(3-azabicyclo[3.3.0]oct-3-yl-amino)-8-(2-ethoxyethyl)pyrido[2,3-d]pyrimidin-7(8H)-one (Compound No. 25),
2-[6-(2-Chlorophenyl)-2-(3-azabicyclo[3.3.0]oct-3-yl-amino)-7-oxopyrido[2,3-d]pyrimidin-8(7H)-yl]ethyl acetate (Compound No. 26),
6-(2-Chlorophenyl)-2-(3-azabicyclo[3.3.0]oct-3-yl-amino)-8-(2-methoxyethyl)pyrido[2,3-d]pyrimidin-7(8H)-one (Compound No. 27),
6-(2-Methylphenyl)-2-(3-azabicyclo[3.3.0]oct-3-yl-amino)-8-[2-(2-methoxyethoxy)ethyl]-pyrido[2,3-d]pyrimidin-7(8H)-one (Compound No. 28),
6-(2-Chlorophenyl)-2-(3-azabicyclo[3.3.0]oct-3-yl-amino)-8-[2-(2-methoxyethoxy)ethyl]pyrido[2,3-d]pyrimidin-7(8H)-one (Compound No. 29),
Tert-butyl 4-{[2-(3-azabicyclo [3.3.0]oct-3-yl-amino)-6-(2-methylphenyl)-7-oxopyrido[2,3-d]pyrimidin-8(7H)-yl]methyl}piperidine-1-carboxylate (Compound No. 32),
2-{2-[2-(3-Azabicyclo[3.3.0]oct-3-yl-amino)-6-(2-methylphenyl)-7-oxopyrido[2,3-d]pyrimidin-8(7H)-yl]ethyl}-1H-isoindole-1,3(2H)-dione (Compound No. 34),
Ethyl[2-(3-azabicyclo[3.3.0]oct-3-yl-amino)-6-(2-methylphenyl)-7-oxopyrido[2,3-d]pyrimidin-8(7H)-yl]acetate (Compound No. 44).

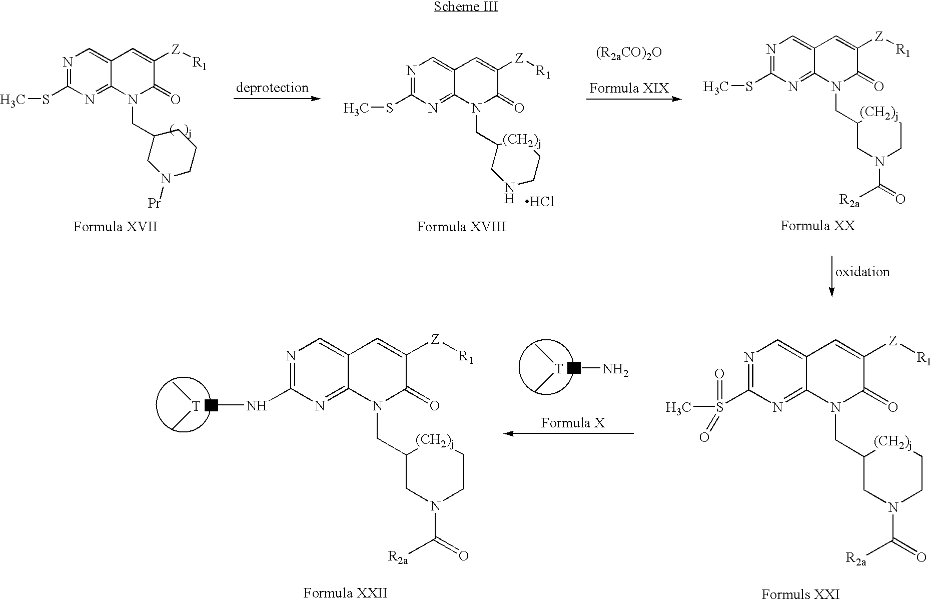

Compounds of Formula XXII can be and were prepared following the procedure as depicted in Scheme III. An exemplary reaction can comprise deprotecting a compound of Formula XVII (wherein j is an integer from 0-2 and Pr is protecting group for example, —C(═O)OC(CH₃)₃, —C(═O)OC(CH₃)₂CHBr₂ or —C(═O)OC(CH₃)₂CCl₃) to give a compound of Formula XVIII, which can be reacted with a compound of Formula XIX (wherein R₂ₐ and hal are the same as defined earlier) to give a compound of Formula XX, which can be oxidized to give a compound of Formula XXI, which can be reacted with a compound of Formula X to give a compound of Formula XXII.

The deprotection of a compound of Formula XVII (wherein Pr can be —C(═O)OC(CH₃)₃ or —C(═O)OC(CH₃)₂CHBr₂) to give a compound of Formula XVIII can be carried out in an acidic solution of an alcohol (for example, hydrochloric acid solution of methanol, ethanol, propanol, isopropylalcohol, ethylacetate or ether) or trifluoroacetic acid in dichloromethane.

The deprotection of a compound of Formula XVII (wherein Pr can be —C(═O)OC(CH₃)₂CCl₃) to give a compound of Formula XVIII can be carried out by a supemucleophile (for example, lithium cobalt (I) phthalocyanine, zinc and acetic acid or cobalt phthalocyanine).

The reaction of a compound of Formula XVIII with a compound of Formula XIX to give a compound of Formula XX can be carried out in the presence of a base, for example, pyridine, N-methylmorpholine or diisopropylethylamine.

The oxidation of a compound of Formula XX to give a compound of Formula XXI can be carried out with an oxidizing agent, for example, m-chloroperbenzoic acid or oxone (KHSO₅), in an organic solvent, for example, chloroform, carbon tetrachloride, dichloromethane, ethanol or tetrahydrofuran.

The reaction of a compound of Formula XXI with a compound of Formula X to give a compound of Formula XXII can be carried out in the presence of a base, for example, pyridine, N-methylmorpholine, N-ethyldiisopropylamine, sodium hydride or triethylamine.

Illustrative compounds include that mentioned below:
6-(2-Methylphenyl)-2-(3-azabicyclo[3.3.0]oct-3-yl-amino)-8-[(1-acetylpiperidin-4-yl)methyl]-pyrido[2,3-d]pyrimidin-7(8H)-one (Compound No. 33).

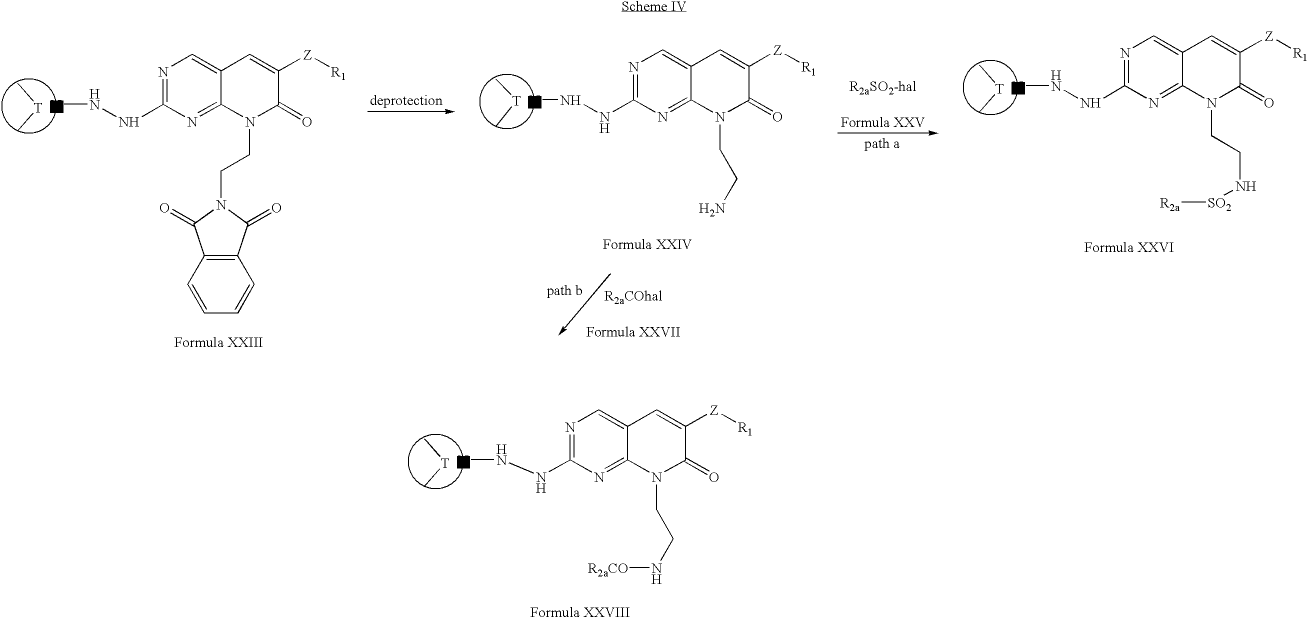

Compounds of Formulae XXVI and XXVIII can be and were prepared following a procedure, such as that depicted in Scheme IV. An exemplary reaction can comprise deprotecting a compound of Formula XXIII (wherein Z and R₁ are the same as defined earlier) to give a compound of Formula XXIV,

Path a: reacting a compound of Formula XXIV with a compound of Formula XXV (wherein R₂ₐ and hal are the same as defined earlier) to give a compound of Formula XXVI.

Path b: reacting a compound of Formula XXIV with a compound of Formula XXVII (wherein R₂ₐ and hal are the same as defined earlier) to give a compound of Formula XXVIII.

The deprotection of a compound of Formula XXIII to give a compound of Formula XXIV can be carried out with, for example, aqueous hydrazine, in an organic solvent, for example, ethanol, methanol, propanol or isopropanol.

The reaction of a compound of Formula XXIV with a compound of Formula XXV to give a compound of Formula XXVI can be carried out in the presence of a base, for example, triethylamine, N-methylmorpholine, diisopropylethylamine or pyridine, in an organic solvent, for example, dichloromethane, dichloroethane, chloroform or carbon tetrachloride.

The reaction of a compound of Formula XXIV with a compound of Formula XXVII to give a compound of Formula XXVIII can be carried out in the presence of a base, for example, triethylamine, N-methylmorpholine, diisopropylethylamine or pyridine, in an organic solvent, for example, dichloromethane, dichloroethane, chloroform or carbon tetrachloride.

Illustrative compounds include those mentioned below:
6-(2-Methylphenyl)-2-(3-azabicyclo[3.3.0]oct-3-yl-amino)-8-(2-aminoethyl)-pyrido[2,3-d]pyrimidin-7(8h)-one (Compound No. 35),
N-{2-[2-(3-azabicyclo[3.3.0]oct-3-yl-amino)-6-(2-methylphenyl)-7-oxopyrido[2,3-d]pyrimidin-8(7H)-yl]ethyl}acetamide (Compound No. 36),
N-{2-[2-(3-azabicyclo[3.3.0]oct-3-yl-amino)-6-(2-methylphenyl)-7-oxopyrido[2,3-d]pyrimidin-8(7H)-yl]ethyl}propanamide (Compound No. 37),
N-{2-[2-(3-azabicyclo[3.3.0]oct-3-yl-amino)-6-(2-methylphenyl)-7-oxopyrido[2,3-d]pyrimidin-8(7H)-yl]ethyl}methanesulfonamide (Compound No. 38),
N-{2-[2-(3-azabicyclo[3.3.0]oct-3-yl-amino)-6-(2-methylphenyl)-7-oxopyrido[2,3-d]pyrimidin-8(7H)-yl]ethyl}propane-1-sulfonamide (Compound No. 39),
N-{2-[2-(3-azabicyclo[3.3.0]oct-3-yl-amino)-6-(2-methylphenyl)-7-oxopyrido[2,3-d]pyrimidin-8(7H)-yl]ethyl}butane-1-sulfonamide (Compound No. 40).

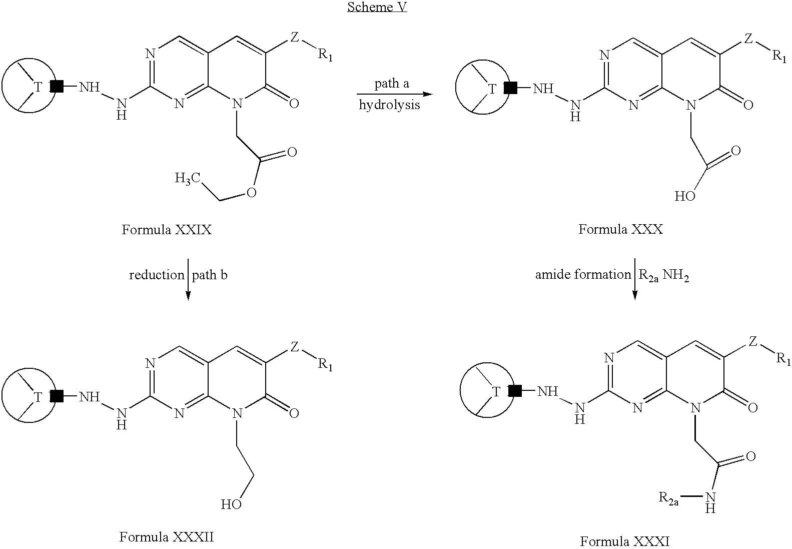

Compounds of Formulae XXX, XXXI and XXXII can be and were prepared following procedures, for example, as depicted in Scheme V. Exemplary reactions can comprise hydrolyzing a compound of Formula XXIX (Path a) (wherein Z, R₁, T are the same as defined earlier) to give a compound of Formula XXX, which can be reacted with a compound of Formula R₂ₐNH₂ (wherein R₂ₐ is the same as defined earlier) to compound of Formula XXXI. The compound of Formula XXIX can be reduced to give a compound of Formula XXXII.

The hydrolysis of the compound of Formula XXIX (path a) to give a compound of Formula XXX can be carried out in the presence of a base, for example, lithium hydroxide, potassium hydroxide or sodium hydroxide, in an organic solvent, for example, methanol, ethanol, propanol, tetrahydrofuran or mixtures thereof.

The compound of Formula XXX can be converted to a compound of Formula XXXI in the presence of a base, for example, N-methylmorpholine, triethylamine, diisopropylethylamine or pyridine, in an organic solvent, for example, tetrahydrofuran, dimethylformamide, diethyl ether or dioxane, with a condensing agent, for example, 1-(3-dimethylaminopropyl)-3-ethylcarbodiimide hydrochloride (EDC.HCl) or dicyclohexylcarbodiimide (DCC).

The reduction of the compound of Formula XXIX (path b) to give a compound of Formula XXXII can be carried out in the presence of reducing agent, for example, sodium borohydride, lithium borohydride or sodium cyanoborohydride.

Particular illustrative compounds include those mentioned below:
6-(2-Methylphenyl)-2-(3-azabicyclo[3.3.0]oct-3-yl-amino)-8-(2-hydroxyethyl)pyrido[2,3-d]pyrimidin-7(8H)-one (Compound No. 45),
6-(2-Methylphenyl)-2-(3-azabicyclo[3.3.0]oct-3-yl-amino)-7-oxopyrido[2,3-d]pyrimidin-8(7H)-yl-N-methoxyacetamide (Compound No. 46),
N-Cyclobutyl-2-[2-(3-azabicyclo[3.3.0]oct-3-yl-amino)-6-(2-methylphenyl)-7-oxopyrido[2,3-d]pyrimidin-8(7H)-yl]acetamide (Compound No. 47),
6-(2-Methylphenyl)-2-(3-azabicyclo[3.3.0]oct-3-yl-amino)-8-(2-oxo-2-pyrrolidin-1-ylethyl)pyrido[2,3-d]pyrimidin-7(8H)-one (Compound No. 48),
6-(2-Methylphenyl)-2-(3-azabicyclo[3.3.0]oct-3-yl-amino)-7-oxopyrido[2,3-d]pyrimidin-8(7H)-yl-N-isopropylacetamide (Compound No. 49),
6-(2-Methylphenyl)-2-(3-azabicyclo[3.3.0]oct-3-yl-amino)-7-oxopyrido[2,3-d]pyrimidin-8(7H)-yl]acetic acid (Compound No. 79),
N-Cyclopropyl-2-[2-(3-azabicyclo[3.3.0]oct-3-yl-amino)-6-(2-methylphenyl)-7-oxopyrido[2,3-d]pyrimidin-8(7H)-yl]acetamide (Compound No. 80).

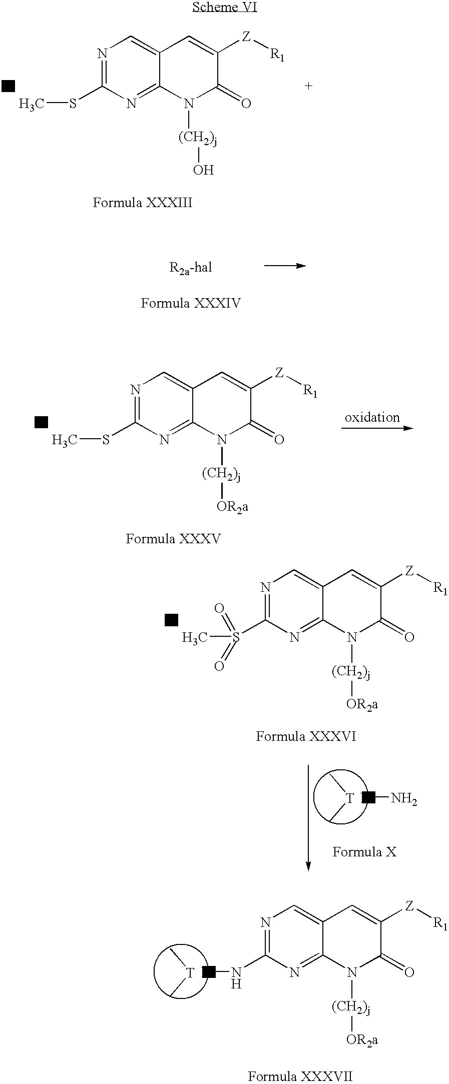

Compounds of Formulae XXXVII can be prepared following, for example, a procedure as depicted in Scheme VI. Thus, an exemplary reaction can comprise reacting a compound of Formula XXXIII (wherein j is an integer from 0-2, and Z, R₁ and T are the same as defined earlier) with a compound of Formula XXXIV (wherein R₂ₐ and hal are the same as defined earlier) to give a compound of Formula XXXV, which can be oxidized to give a compound of Formula XXXVI, which can be reacted with a compound of Formula X to give a compound of Formula XXXVII.

The reaction of a compound of Formula XXXIII with a compound of Formula XXXIV to give a compound of Formula XXXV can be carried out in the presence of a base, for example, sodium hydride, potassium hydride or lithium hydride, in an organic solvent, for example, N-methylpyrrolidone, dimethylformamide, tetrahydrofuran or dimethylsulphoxide.

The oxidation of a compound of Formula XXXV to give a compound of Formula XXXVI can be carried out with an oxidizing agent, for example, m-chloroperbenzoic acid or oxone (KHSO₅), in an organic solvent, for example, chloroform, dichloroethane, carbon tetrachloride, dichloromethane, ethanol or tetrahydrofuran.

The reaction of a compound of Formula XXXVI with a compound of Formula X to give a compound of Formula XXXVII can be carried in the presence of a base, for example, pyridine, N-methylmorpholine, N-ethyldiisopropylamine, sodium hydride or triethylamine.

Particular illustrative compounds include those mentioned below
6-(2-Methylphenyl)-2-(3-azabicyclo[3.3.0]oct-3-yl-amino)-8-(2-methoxyethyl)-pyrido[2,3-d]pyrimidin-7(8H)-one (Compound No. 50),
6-(2-Methylphenyl)-2-(3-azabicyclo[3.3.0]oct-3-yl-amino)-8-(2-ethoxyethyl)pyrido[2,3-d]pyrimidin-7(8H)-one (Compound No. 51).

Particular illustrative compounds which can be produced by Scheme I, are listed in table below:

| Com- | | Com- | |
|---|---|---|---|
| pound | | pound | |
| No | Structure | No. | Structure |
| 1. | 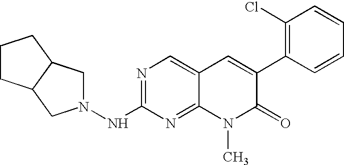 | 2. | 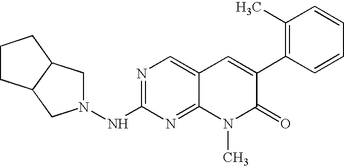 |
| 3. | 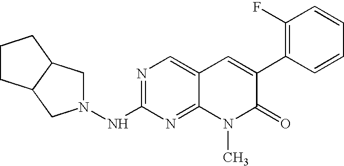 | 4. | 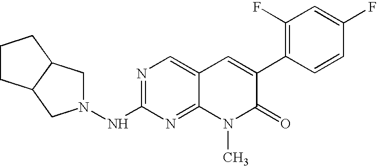 |
| 5. | 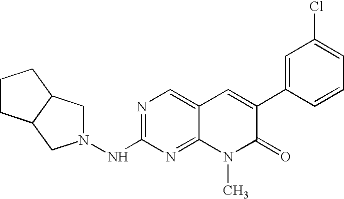 | 6. | 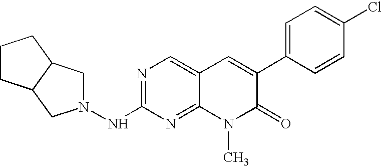 |
| 7. | 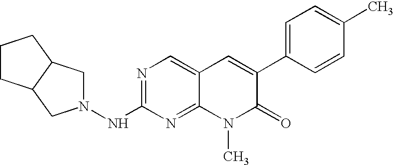 | 8. | 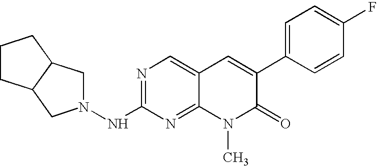 |
| 9. | 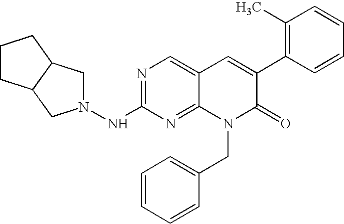 | 10. | 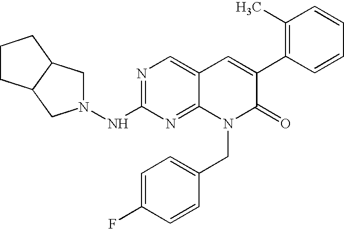 |
| 11. | 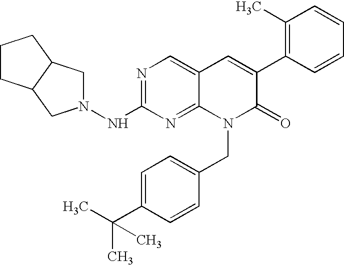 | 12. | 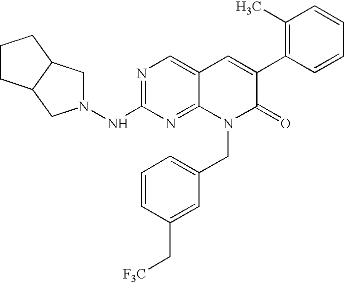 |
| 13. | 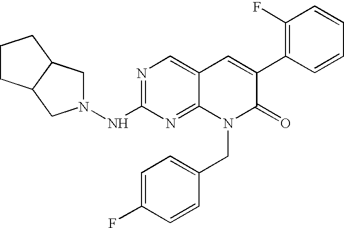 | 14. | 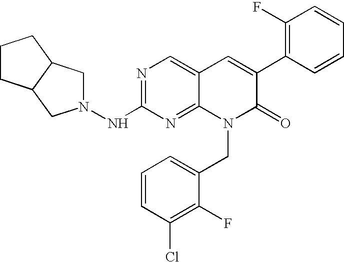 |
| 15. | 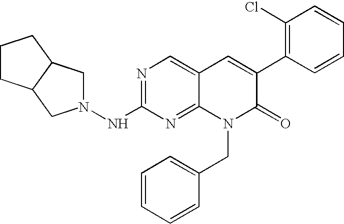 | 16. | 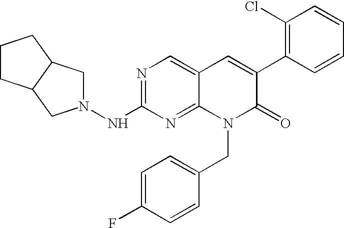 |
| 17. | 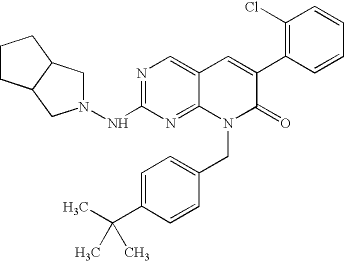 | 18. | 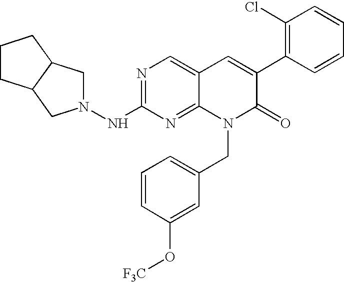 |
| 19 | 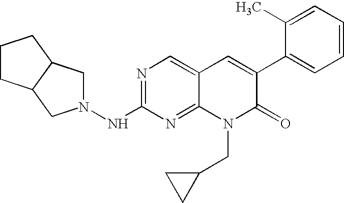 | 20 | 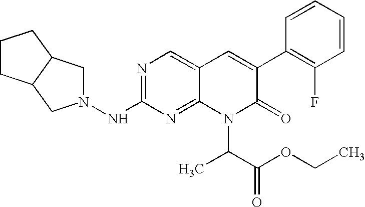 |
| 21 | 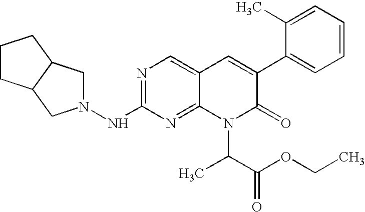 | 22 | 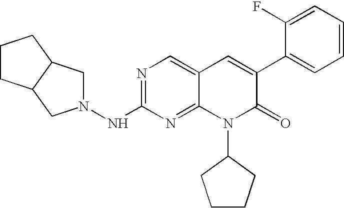 |
| 23 | 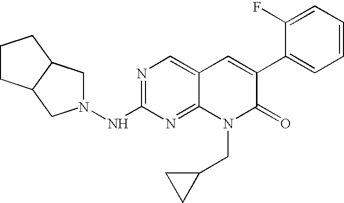 | 24 | 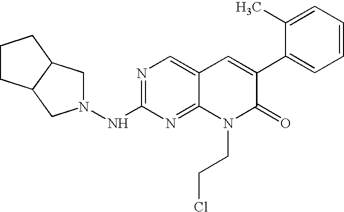 |
| 25 | 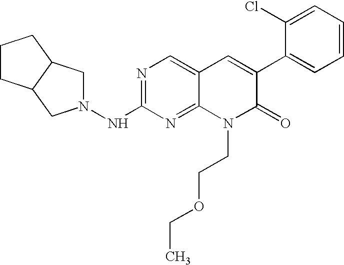 | 26 | 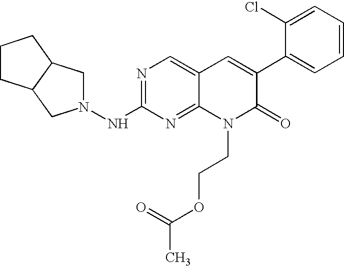 |
| 27. | 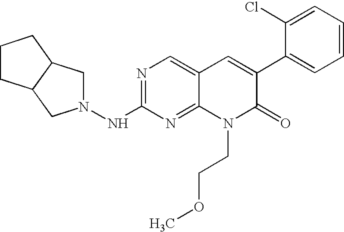 | 28 | 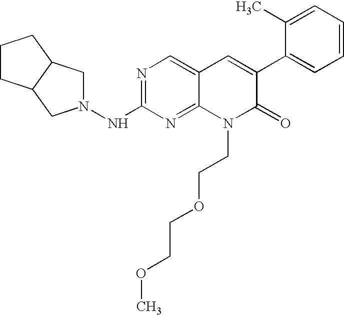 |
| 29. | 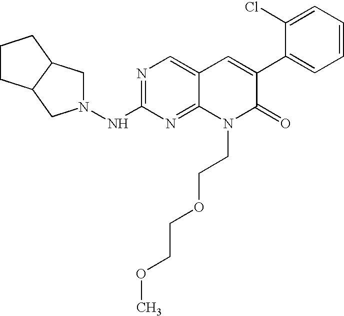 | 30. | 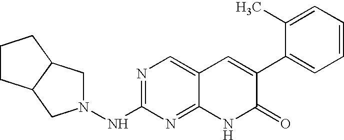 |
| 31. | 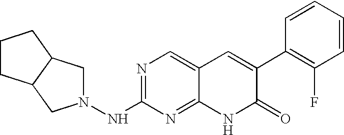 | 32. | 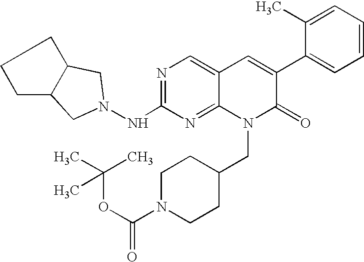 |
| 33. | 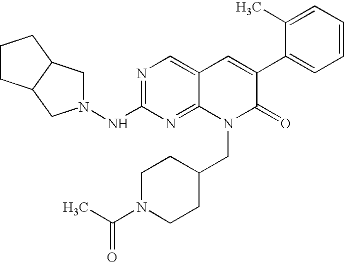 | 34. | 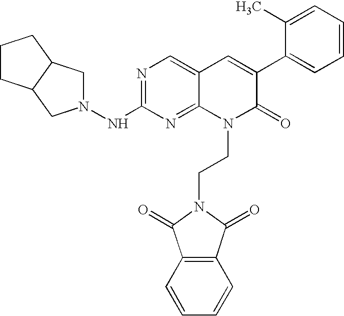 |
| 35. | 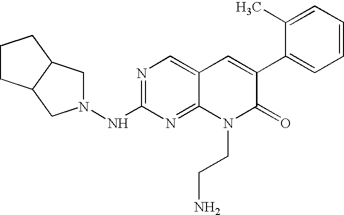 | 36. | 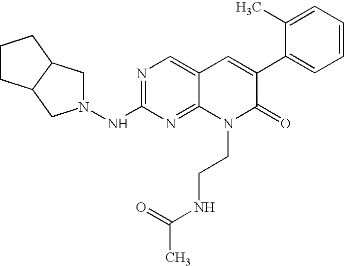 |
| 37. | 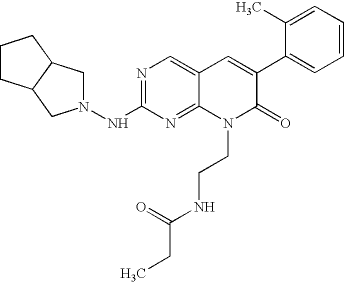 | 38. | 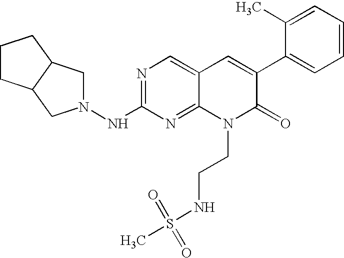 |
| 39. | 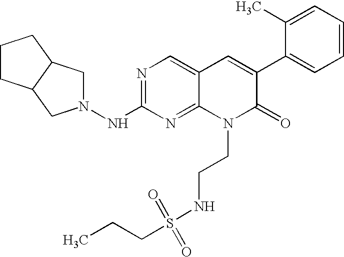 | 40 | 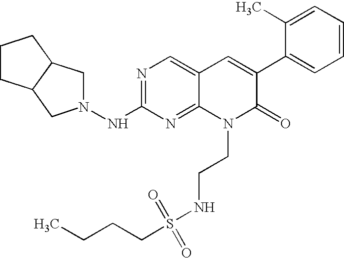 |
| 41. | 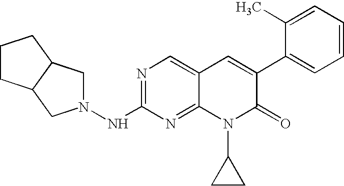 | 42. | 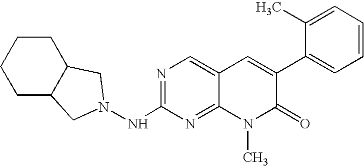 |
| 43. | 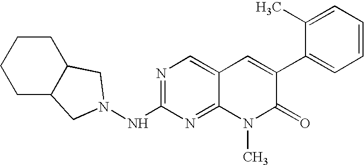 | 44 | 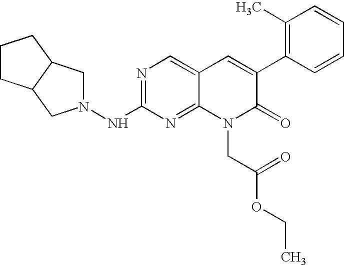 |
| 45. | 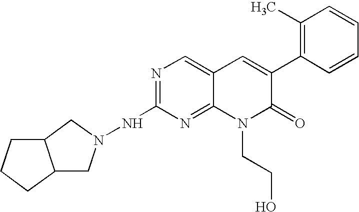 | 46. | 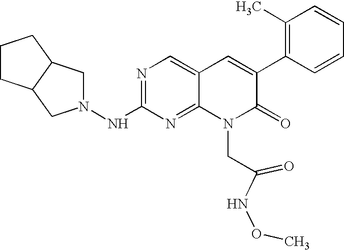 |
| 47 | 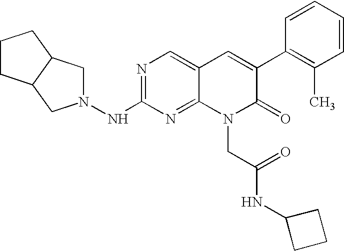 | 48. | 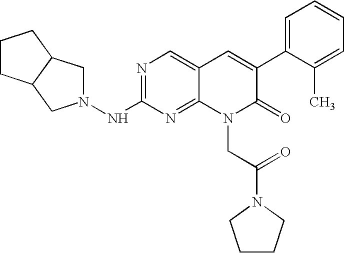 |
| 49. | 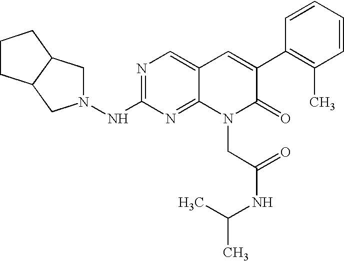 | 50. | 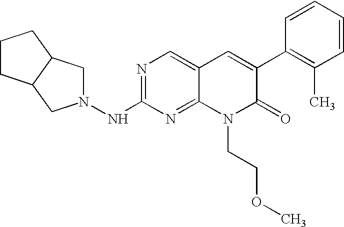 |
| 51. | 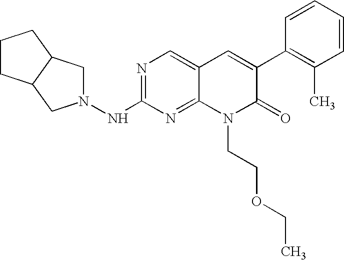 | 52. | 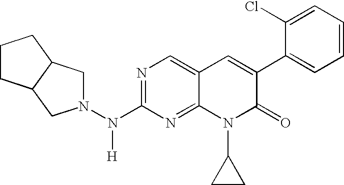 |
| 53. | 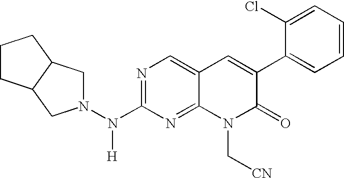 | 54. | 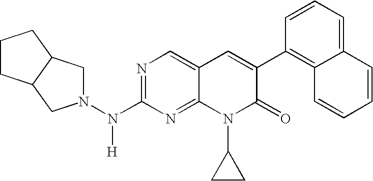 |
| 55. | 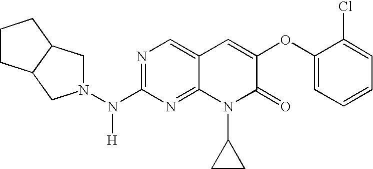 | 56. | 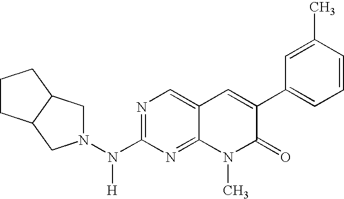 |
| 57. | 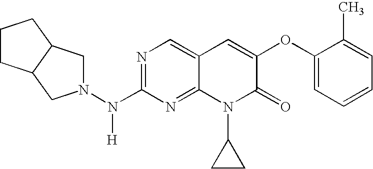 | 58. | 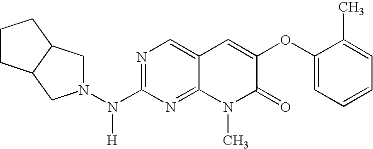 |
| 59. | 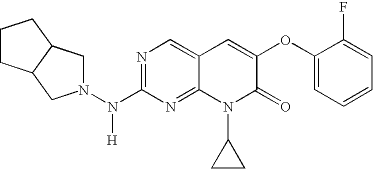 | 60. | 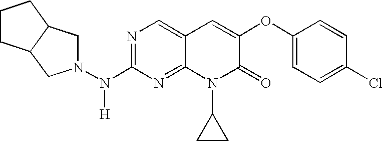 |
| 61. | 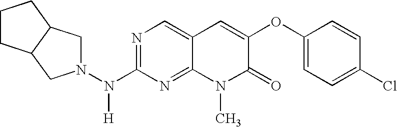 | 62 | 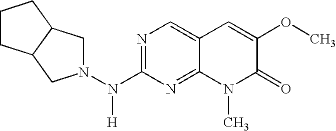 |
| 63. | 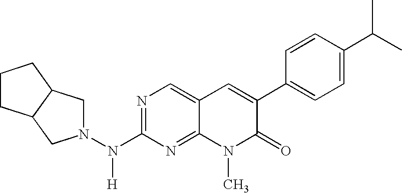 | 64. | 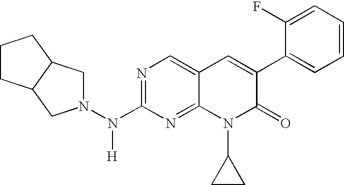 |
| 65. | 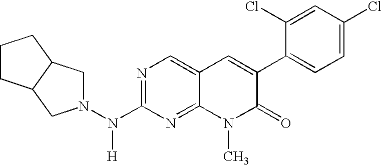 | 66. | 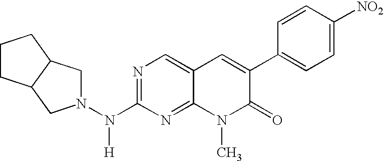 |
| 67. | 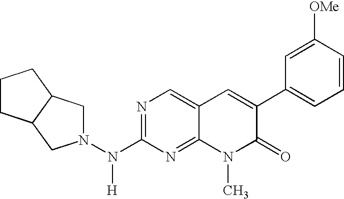 | 68. | 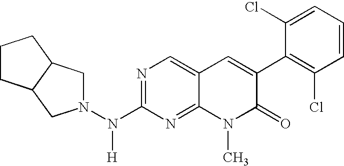 |
| 69. | 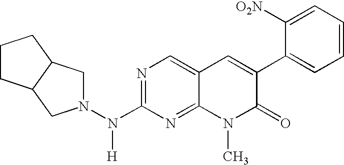 | 70. | 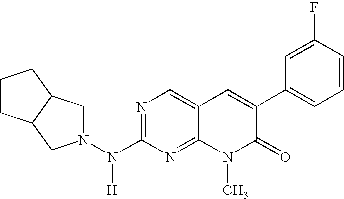 |
| 71. | 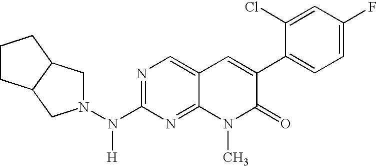 | 72. | 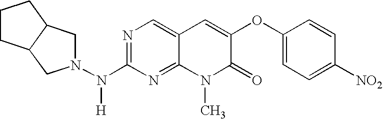 |
| 73. | 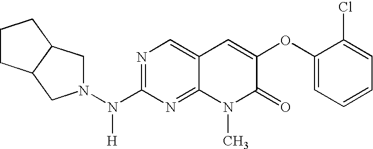 | 74. | 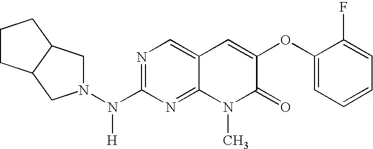 |
| 75. | 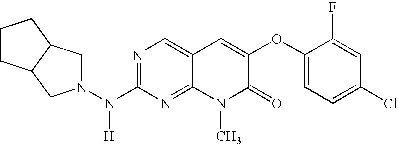 | 76. | 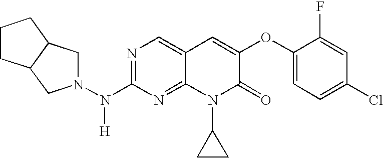 |
| 77 | 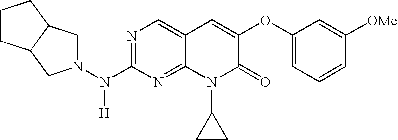 | 78. | 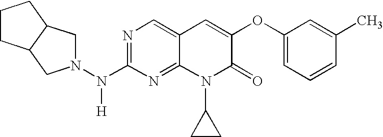 |
| 79 | 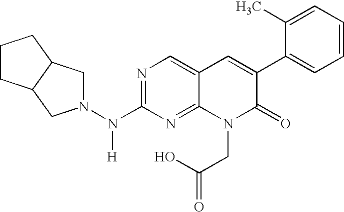 | 80 | 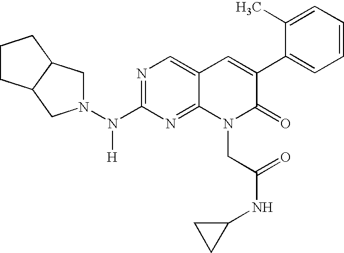 |

Examples set forth herein can demonstrate general synthetic procedures for the preparation of some representative compounds. The examples are provided to illustrate particular aspect of the disclosure and do not limit the scope of the present invention

### EXAMPLES

### Example 1

### Synthesis of 6-(2-chlorophenyl)-2-(3-azabicyclo[3.3.0]oct-3-yl-amino)-8-methylpyrido[2,3-d]pyrimidin-7(8H)-one (Compound No. 1)

### Step a: 4-Methylamino-2-methylthio-pyrimidine-5-carboxylic acid ethyl ester

To a suspension of ethyl-4-chloro-2-methylthio-5-pyrimidine-carboxylate (commercially available) (8.0 g, 34 mmol) in dry tetrahydrofuran (60 ml), was added triethylamine (4.3 g, 42 mmol) and aqueous methylamine (40%, 3.2 g, 36.2 mmol) at room temperature and stirred for 2 hours. The organic solvent was evaporated under reduced pressure followed by addition of cold water. A white solid thus obtained was filtered, washed with water and dried under vacuum. Yield=6.2 g.

### Step b: [4-(Methylamino)-2-(methylthio)-pyrimidin-5-yl]-methanol

To a suspension of lithium aluminium hydride (1.21 g, 32 mmol) in dry tetrahydrofuran (60 ml) at −70° C., was added a solution of the compound obtained from step a above (6.0 g, 26 mmol) in tetrahydrofuran (20 ml) dropwise. The reaction mixture was stirred between −70° C.-60° C. for 1 hour and then at room temperature till completion. The reaction mixture was cooled to 0° C. and diluted with ethylacetate, followed by addition of 30% aqueous solution of sodium hydroxide dropwise. The reaction mixture was then filtered through a celite pad and washed with ethylacetate and dichloromethane. The filtrate was evaporated under reduced pressure followed by addition of water. A white solid thus obtained was filtered and dried under vacuum. Yield=4.0 g.

### Step c: 4-Methylamino-2-methylthio-pyrimidin-5-carboxaldehyde

To a suspension of compound obtained from step b above (3.8 g, 20.7 mmol) in dichloromethane (100 ml), was added manganese dioxide (12.7 g, 145 mmol) at room temperature and stirred for 24-36 hours. The reaction mixture was filtered over a celite pad and evaporated under reduced pressure. The residue thus obtained was purified by column chromatography using ethylacetate in hexane (1:4) solvent mixture as eluent to furnish the title compound. Yield=3.2 g. A particular exemplary compound was prepared analogously, namely, 4-Cyclopropylamino-2-methylthio-pyrimidin-5-carboxaldehyde.

### Step d: 6-(2-Chlorophenyl)-8-methyl-2-methylthio-8H-pyrido[2,3-d]pyrimidin-7-one

To a solution of the compound obtained from step c above (3.2 g, 17.7 mmol) in N-methylpyrrolidinone (20 ml), was added 2-chlorophenyl acetic acid methyl ester (4.9 g, 26.6 mmol) and potassium carbonate (7.4 g, 53.04 mmol) and heated at 110° C. for 2 hours. The reaction mixture was diluted with ethylacetate and poured into water. It was then extracted with ethyl acetate, washed with water, dried over anhydrous sodium sulphate, filtered and evaporated under reduced pressure. The residue thus obtained was purified by column chromatography using ethylacetate in hexane (1:3) as eluent. Yield=3.2 g.

### Step e: 6-(2-Chlorophenyl)-2-methanesulphonyl-8-methyl-8H-pyrido[2,3-d]pyrimidin-7-one

To a solution of the compound obtained from step d above (1.5 g, 4.7 mmol) in chloroform (20 ml) was added m-chloroperbenzoic acid (70%) (3.5 g, 14.2 mmol) at 0° C. and stirred at room temperature for 30 minutes. To it was added a saturated solution of aqueous sodium bisulphate followed by aqueous sodium bicarbonate solution at 0° C. The reaction mixture was then extracted with dichloromethane and the organic layer was washed with water, dried over anhydrous sodium sulphate, filtered and evaporated under reduced pressure. The residue thus obtained was washed thoroughly with hexane to furnish the title compound. Yield=1.1 g

### Step f: 6-(2-Chlorophenyl)-2-(3-azabicyclo [3.3.0]oct-3-yl-amino)-8-methylpyrido[2,3-d]pyrimide-7(8H)-one

To the compound obtained from step e above (0.1 g, 0.286 mmol), was added 3-amino-3-azabicyclo[3.3.0]octane (commercially available) (0.116 g, 0.715 mmol) and heated to 80° C. for 2 hours. The reaction mixture was diluted with dichloromethane and the compound was purified by column chromatography using (1:1) ethyl acetate:hexane as eluent to afford a yellow residue which was then further purified by preparative TLC using ethyl acetate in dichloromethane as solvent of elution. Yield=35 mg. m.p.: 85-87° C.

¹H NMR (CDCl₃): δ 8.55 (1H, s, Ar—H), 7.54 (1H, s, Ar—H), 7.48-7.44 (1H, t, J=3.0 Hz, Ar—H), 7.35-7.30 (3H, m, Ar—H), 3.72 (3H, s, —NCH₃), 3.32 (2H, brs, —NCH₂), 2.74 (2H, brs, —NCH₂), 2.49 (2H, brs, −2x-CH) and 1.73-1.53 (6H, m, 3x-CH₂).

Mass spectrum (m/z, +ve ion mode): 398 [M⁺+1+2] and 396 [M⁺+1].

Analogues of 6-(2-chlorophenyl)-2-(3-azabicyclo[3.3.0]oct-3-yl-amino)-8-methylpyrido[2,3-d]pyrimidin-7(8H)-one (Compound No. 1) described below were prepared by using appropriate amine in place of 3-amino-3-azabicyclo[3.3.0]octane, respectively, as applicable in each case.
6-(2-Methylphenyl)-2-(3-azabicyclo[3.3.0]oct-3-yl-amino)-8-methylpyrido[2,3-d]pyrimidin-7(8H)-one (Compound No. 2)

m.p: 69.8-79.8° C.; ¹H NMR (CDCl₃+CD₃OD): δ 9.09 (s, 1H, Ar—H), 7.52 (s, 1H, Ar—H), 7.33-7.17 (m, 4H, Ar—H), 3.74 (s, 3H, —NCH₃), 2.63-2.56 (m, 6H, 2x-NCH₂ & 2x-CH), 2.21 (s, 3H, —ArCH₃) and 1.73-1.70 (m, 6H, 3x-CH₂); Mass spectrum (m/z, +ve ion mode): 376 [M⁺+1].
6-(2-Fluorophenyl)-2-(3-azabicyclo[3.3.0]oct-3-yl-amino)-8-methylpyrido[2,3-d]pyrimidin-7(8H)-one (Compound No. 3),
6-(2,4-Difluorophenyl)-2-(3-azabicyclo[3.3.0]oct-3-yl-amino)-8-methylpyrido[2,3-d]pyrimidin-7(8H)-one (Compound No. 4),
6-(3-Chlorophenyl)-2-(3-azabicyclo[3.3.0]oct-3-yl-amino)-8-methylpyrido[2,3-d]pyrimidin-7(8h)-one (Compound No. 5),
6-(4-Chlorophenyl)-2-(3-azabicyclo[3.3.0]oct-3-yl-amino)-8-methylpyrido[2,3-d]pyrimidin-7(8H)-one (Compound No. 6),
6-(4-Methylphenyl)-2-(3-azabicyclo[3.3.0]oct-3-yl-amino)-8-methylpyrido[2,3-d]pyrimidin-7(8H)-one (Compound No. 7),
6-(4-Fluorophenyl)-2-(3-azabicyclo[3.3.0]oct-3-yl-amino)-8-methylpyrido[2,3-d]pyrimidin-7(8H)-one (Compound No. 8),
6-(2-Methylphenyl)-2-(3-azabicyclo[3.3.0]oct-3-yl-amino)-8-(benzyl)-pyrido[2,3-d]pyrimidin-7(8H)-one (Compound No. 9),
6-(2-Methylphenyl)-2-(3-azabicyclo[3.3.0]oct-3-yl-amino)-8-(4-fluorobenzyl)-pyrido[2,3-d]pyrimidin-7(8H)-one (Compound No. 10),
6-(2-Methylphenyl)-2-(3-azabicyclo[3.3.0]oct-3-yl-amino)-8-(tert-butylbenzyl)-pyrido[2,3-d]pyrimidin-7(8h)-one (Compound No. 11),
6-(2-Methylphenyl)-2-(3-azabicyclo[3.3.0]oct-3-yl-amino)-8-(3-trifluoromethoxybenzyl)-pyrido[2,3-d]pyrimidin-7(8H)-one (Compound No. 12),
6-(2-Fluorophenyl)-2-(3-azabicyclo[3.3.0]oct-3-yl-amino)-8-(4-fluorobenzyl)-pyrido[2,3-d]pyrimidin-7(8H)-one (Compound No. 13),
6-(2-Fluorophenyl)-2-(3-azabicyclo[3.3.0]oct-3-yl-amino)-8-(3-chloro-2-fluorobenzyl)-pyrido[2,3-d]pyrimidin-7(8H)-one (Compound No. 14),
6-(2-Chlorophenyl)-2-(3-azabicyclo[3.3.0]oct-3-yl-amino)-8-(benzyl)-pyrido[2,3-d]pyrimidin-7(8H)-one (Compound No. 15),
6-(2-Chlorophenyl)-2-(3-azabicyclo[3.3.0]oct-3-yl-amino)-8-(4-fluorobenzyl)-pyrido[2,3-d]pyrimidin-7(8H)-one (Compound No. 16),
6-(2-Chlorophenyl)-2-(3-azabicyclo[3.3.0]oct-3-yl-amino)-8-(4-tert-butylbenzyl)-pyrido[2,3-d]pyrimidin-7(8H)-one (Compound No. 17),
6-(2-Chlorophenyl)-2-(3-azabicyclo[3.3.0]oct-3-yl-amino)-8-(3-trifluoromethoxybenzyl)-pyrido[2,3-d]pyrimidin-7(8H)-one (Compound No. 18),
6-(2-Methylphenyl)-2-(3-azabicyclo[3.3.0]oct-3-yl-amino)-pyrido[2,3-d]pyrimidin-7(8H)-one (Compound No. 30),
6-(2-Fluorophenyl)-2-(3-azabicyclo[3.3.0]oct-3-yl-amino)pyrido[2,3-d]pyrimidin-7(8H)-one (Compound No. 31),
6-(2-Methylphenyl)-2-(7-azabicyclo[4.3.0]non-7-yl-amino)-8-(cyclopropyl)-pyrido[2,3-d]pyrimidin-7(8H)-one (Compound No. 41),
6-(2-Methylphenyl)-2-(7-azabicyclo[4.3.0]non-7-yl-amino)-8-methyl-pyrido[2,3-d]pyrimidin-7(8H)-one (Compound No. 42),
6-(2-Chlorophenyl)-2-(7-azabicyclo[4.3.0]non-7-yl-amino)-8-methyl-pyrido[2,3-d]pyrimidin-7(8H)-one (Compound No. 43),
6-(2-Chlorophenyl)-2-(3-azabicyclo[3.3.0]oct-3-yl-amino)-8-cyclopropyl-pyrido[2,3-d]pyrimidin-7(8H)-one (Compound No. 52),
6-(2-Chlorophenyl)-2-(3-azabicyclo[3.3.0]oct-3-yl-amino)-7-oxopyrido[2,3-d]pyrimidin-8(7H)-ylacetonitrile (Compound No. 53),
6-(1-Naphthyl)-2-(3-azabicyclo[3.3.0]oct-3-yl-amino)-8-cyclopropyl-pyrido[2,3-d]pyrimidin-7(8H)-one (Compound No. 54),
6-(3-Methylphenyl)-2-(3-azabicyclo[3.3.0]oct-3-yl-amino)-8-methyl-pyrido[2,3-d]pyrimidin-7(8H)-one (Compound No. 56),
6-(4-Isopropylphenyl)-2-(3-azabicyclo[3.3.0]oct-3-yl-amino)-8-methylpyrido[2,3-d]pyrimidin-7(8H)-one (Compound No. 63),
6-(2-Fluorophenyl)-2-(3-azabicyclo[3.3.0]oct-3-yl-amino)-8-(cyclopropyl)pyrido[2,3-d]pyrimidin-7(8H)-one (Compound No. 64),
6-(2,4-Dichlorophenyl)-2-(3-azabicyclo[3.3.0]oct-3-yl-amino)-8-methylpyrido[2,3-d]pyrimidin-7(8H)-one (Compound No. 65),
6-(4-Nitrophenyl)-2-(3-azabicyclo[3.3.0]oct-3-yl-amino)-8-methyl-pyrido[2,3-d]pyrimidin-7(8H)-one (Compound No. 66),
6-(3-Methoxyphenyl)-2-(3-azabicyclo[3.3.0]oct-3-yl-amino)-8-methylpyrido[2,3-d]pyrimidin-7(8H)-one (Compound No. 67),
6-(2,6-Dichlorophenyl)-2-(3-azabicyclo[3.3.0]oct-3-yl-amino)-8-methylpyrido[2,3-d]pyrimidin-7(8H)-one (Compound No. 68),
6-(2-Nitrophenyl)-2-(3-azabicyclo[3.3.0]oct-3-yl-amino)-8-methyl-pyrido[2,3-d]pyrimidin-7(8H)-one (Compound No. 69),
6-(3-Fluorophenyl)-2-(3-azabicyclo[3.3.0]oct-3-yl-amino)-8-methylpyrido[2,3-d]pyrimidin-7(8H)-one (Compound No. 70),
6-(2-Chloro-4-fluorophenyl)-2-(3-azabicyclo[3.3.0]oct-3-yl-amino)-8-methylpyrido[2,3-d]pyrimidin-7(8H)-one (Compound No. 71),

### Example 1a

### Synthesis of 6-(2-fluorophenoxy)-2-(3-azabicyclo[3.3.0]oct-3-yl-amino)-8-cyclopropyl-pyrido[2,3-d]pyrimidin-7(8H)-one (Compound No. 59)

### Step a: 1-(2-Fluorophenoxy)acetic acid ethyl ester

To a solution of 2-fluorophenol (5.0 g, 44.64 mmol) in N-methylpyrrolidone (20 ml) added potassium carbonate (18.51 g, 133.92 mmol) and stirred for 10 minutes. To it ethylbromoacetate (8.95 g, 53.56 mmol) was added and the contents were stirred at room temperature overnight. The reaction mixture was poured into water, extracted with ethyl acetate, washed with water, dried over anhydrous sodium sulphate, filtered and the solvent evaporated under vacuum to furnish the title compound.

### Step b: 8-Cyclopropyl-6-(2-fluorophenoxy)-2-(methylthio)-8H-pyrido[2,3-d]pyrimidin-7-one

To a solution of the compound 4-cyclopropylamino-2-methylthio-pyrimidin-5-carboxldehyde (1.0 g, 4.78 mmol) in N-methylpyrrolidone (10 ml) was added potassium carbonate (1.98 g, 14.35 mmol) and stirred at room temperature for 10 min. To it was added the compound obtained from step a above (1.14 g, 5.74 mmol) and the contents were stirred at 110° C. for 5 hrs. The reaction mixture was poured into water and stirred. The solid thus separated out was filtered and dried under vacuum to furnish the title compound.

### Step c: 8-Cyclopropyl-6-(2-fluorophenoxy)-2-(methylsulphonyl)-8H-pyrido[2,3-d]pyrimidin-7-one

The title compound was prepared following the procedure as described in Example 1, step e.

### Step d: 6-(2-Fluorophenoxy)-2-(3-azabicyclo[3.3.0]oct-3-yl-amino)-8-cyclopropyl-pyrido[2,3-d]pyrimidin-7(8H)-one (Compound No. 59)

A solution of the compound 3-amino-3-azabicyclo[3.3.0]octane (commercially available) (0.390 g, 2.40 mmol) in Hunig's base (1.4 ml, 8.0 mmol) was stirred at room temperature for 15 min. To it was added a solution of the compound obtained from step c above (0.30 g, 8.0 mmol) and heated at 110° C. for 1 hr. The reaction mixture was poured into water, extracted with ethyl acetate, dried over anhydrous sodium sulphate, filtered and evaporated under vacuum. The residue thus obtained was purified by column chromatography using 20% ethyl acetate in hexane as eluent and finally by preparative TLC using 10% ethyl acetate in dichloromethane as eluent to furnish the title compound.

Yield: 40 mg; ¹H NMR (CDCl₃): δ 8.36 (1H, s, Ar—H), 7.21-6.78 (3H, m, Ar—H), 6.78 (1H, s, Ar—H), 5.90 (1H, brs, —NH), 3.32-3.31 (2H, brm, —NCH₂), 3.00-2.97 (1H, brm, —NCH), 2.74-2.72 (2H, brm, —NCH₂), 2.46 (2H, brm, 2x-CH) and 1.70-1.52 (6H, m, 3x-CH₂), 1.32-1.26 (2H, m, —CH₂) and 0.97-0.96 (2H, m, —CH₂); Mass spectrum (m/z, +ve ion mode): 422 [M⁺+1].

The following illustrative compounds were prepared analogously,
6-(2-Chlorophenoxy)-2-(3-azabicyclo[3.3.0]oct-3-yl-amino)-8-cyclopropyl-pyrido[2,3-d]pyrimidin-7(8h)-one (Compound No. 55)
6-(2-Methylphenoxy)-2-(3-azabicyclo[3.3.0]oct-3-yl-amino)-8-cyclopropyl-pyrido[2,3-d]pyrimidin-7(8H)-one (Compound No. 57)
6-(2-Methylphenoxy)-2-(3-azabicyclo[3.3.0]oct-3-yl-amino)-8-methylpyrido[2,3-d]pyrimidin-7(8H)-one (Compound No. 58)
6-(4-Chlorophenoxy)-2-(3-azabicyclo[3.3.0]oct-3-yl-amino)-8-cyclopropylpyrido[2,3-d]pyrimidin-7(8H)-one (Compound No. 60)
6-(4-Chlorophenoxy)-2-(3-azabicyclo[3.3.0]oct-3-yl-amino)-8-methylpyrido[2,3-d]pyrimidin-7(8h)-one (Compound No. 61)
6-Methoxy-2-(3-azabicyclo[3.3.0]oct-3-yl-amino)-8-methylpyrido[2,3-d]pyrimidin-7(8H)-one (Compound No. 62)
6-(4-Nitrophenoxy)-2-(3-azabicyclo[3.3.0]oct-3-yl-amino)-8-methyl-pyrido[2,3-d]pyrimidin-7(8H)-one (Compound No. 72)
6-(2-Chlorophenoxy)-2-(3-azabicyclo[3.3.0]oct-3-yl-amino)-8-methylpyrido[2,3-d]pyrimidin-7(8H)-one (Compound No. 73)
6-(2-Fluorophenoxy)-2-(3-azabicyclo[3.3.0]oct-3-yl-amino)-8-methylpyrido[2,3-d]pyrimidin-7(8H)-one (Compound No. 74)
6-(4-Chloro-2-fluorophenoxy)-2-(3-azabicyclo[3.3.0]oct-3-yl-amino)-8-methylpyrido[2,3-d]pyrimidin-7(8H)-one (Compound No. 75)
6-(4-Chloro-2-fluorophenoxy)-2-(3-azabicyclo[3.3.0]oct-3-yl-amino)-8-cyclopropyl-pyrido[2,3-d]pyrimidin-7(8H)-one (Compound No. 76)
6-(3-Methoxyphenoxy)-2-(3-azabicyclo [3.3.0]oct-3-yl-amino)-8-(cyclopropyl)-pyrido[2,3-d]pyrimidin-7(8H)-one (Compound No. 77)
6-(3-Methylphenoxy)-2-(3-azabicyclo[3.3.0]oct-3-yl-amino)-8-(cyclopropyl)-pyrido[2,3-d]pyrimidin-7(8H)-one (Compound No. 78).

### Scheme II, Procedure

### Example 2

### Synthesis of 6-(2-methylphenyl)-2-(3-azabicyclo[3.3.0]oct-3-yl-amino)-8-(cyclopropyl methyl)pyrido[2,3-d]pyrimidin-7(8H)-one (Compound No. 19)

### Step a: 6-(2-Methylphenyl)-2-methylthio-8H-pyrido[2,3-d]pyrimidin-7-one

The title compound was prepared following the procedure as described in Example 1, steps a to d, by using ammonia in place of methyl amine in step a and by using 2-methylphenyl acetic acid methyl ester in place of 2-chlorophenyl acetic acid methyl ester.

### Step b: 8-Cyclopropylmethyl-6-(2-methylphenyl)-2-methylthio-8H-pyrido[2,3-d]pyrimidin-7-one

To a suspension of sodium hydride (0.040 g, 0.97 mmol) in dimethylformamide at 0° C. was added the compound obtained from step a above (0.250 g, 0.88 mmol). The reaction mixture was stirred at room temperature for 30 minutes. To it was added cyclopropylmethyl bromide (0.13 g, 0.97 mmol) at 0° C. The reaction mixture was stirred at room temperature for 1 hour. The reaction mixture was then poured in water, extracted with ethyl acetate, washed with water dried over anhydrous sodium sulphate, filtered and evaporated under reduced pressure. The residue thus obtained was then purified by column chromatography using 30% ethyl acetate in hexane to afford the title compound.
Step c: 8-Cyclopropylmethyl-6-(2-Methylphenyl)-2-methanesulphonyl-8H-pyrido[2,3-d]pyrimidin-7-one

The title compound was prepared following the procedure as described in Example 1, step e, by oxidizing the compound obtained from step b above.
Step d: 6-(2-Methylphenyl)-2-(3-azabicyclo [3.3.0]oct-3-yl-amino)-8-(cyclopropyl methyl)pyrido[2,3-d]pyrimidin-7(8H)-one (Compound No. 19)

The title compound was prepared following the procedure as described in Example 1, step f.

Yield: 28 mg; ¹H NMR (CDCl₃): δ 8.53 (1H, s, Ar—H), 7.46 (1H, s, Ar—H), 7.26-7.22 (4H, m, Ar—H), 4.32-4.31 (2H, d, J=4 Hz), 2.72-2.70 (2H, m), 2.24 (3H, s), 1.75-1.72 (4H, m), 1.60-1.53 (6H, m), 1.44-1.40 (3H, m), 1.26 (2H, s); Mass spectrum (m/z, +ve ion mode): 416 [M⁺+1].

### Example 3

### Synthesis of Ethyl 2-[6-(2-fluorophenyl)-2-(3-azabicyclo[3.3.0]oct-3-yl-amino)-7-oxopyrido[2,3-d]pyrimidin-8(7h)-yl]propanoate (Compound No. 20)

### Step a: 6-(2-Fluorophenyl)-2-methylthio-8H-pyrido[2,3-d]pyrimidin-7-one

The title compound was prepared following the procedure as described in Example 1, steps a to d, by using ammonia in place of methyl amine in step a and by using 2-fluorophenyl acetic acid methyl ester in place of 2-chlorophenyl acetic acid methyl ester.

### Step b: 2-[6-(2-Fluorophenyl)-2-methylthio-7-oxo-7H-pyrido[2,3-d]pyrimidin-8-yl]-propionic acid ethyl ester

To a solution of the compound obtained from step a above (0.500 g, 1.7 mmol) in acetone (10 ml) was added potassium carbonate (0.73 g, 5.3 mmol), tetra-n-butylammonium iodide (0.98 g, 2.6 mmol), ethyl-2-bromopropionate (0.39 g, 2.1 mmol) and acetone. The reaction mixture was then refluxed for 8 hours. The reaction mixture was cooled to room temperature and filtered. The filtrate was concentrated to obtain the crude product. Crude product was then purified by column chromatography using 20% ethyl acetate in hexane to furnish the title compound.

### Step c: 2-[6-(2-Fluorophenyl)-2-methylsulphonyl-7-oxo-7H-pyrido[2,3-d]pyrimidin-8-yl]-propionic acid ethyl ester

The title compound was prepared following the procedure as described in Example 1, step e, by oxidizing the compound obtained from step b above.

### Step d: Ethyl 2-[6-(2-fluorophenyl)-2-(3-azabicyclo[3.3.0]oct-3-yl-amino)-7-oxopyrido[2,3-d]pyrimidin-8(7H)-yl]propanoate (Compound No. 20)

The title compound was prepared following the procedure as described in Example 1, step f.

Yield: 300 mg; ¹H NMR (CDCl₃): δ8.59 (1H, s, Ar—H), 7.66 (1H, s, Ar—H), 7.65-7.51 (1H, m, Ar—H), 7.33 (1H, s, Ar—H), 7.20-7.11 (2H, m, Ar—H), 5.95 (1H, s, —NCH), 4.25-4.19 (1H, m, —OCH), 4.12-4.08 (1H, m, —OCH), 3.30 (2H, brs, —NCH₂), 2.72 (2H, brs), 2.4 (2H, brs, —NCH₂), 1.69-1.68 (3H, d, J=4 Hz, —CHCH₃), 1.53 (2H, brs), 1.42-1.41 (2H, m), 1.18-1.16 (3H, t, —OCH₂CH₃), 0.98-0.88 (2H, m); Mass spectrum (m/z, +ve ion mode): 466 [M⁺+1].

The following illustrative compounds were prepared analogously,
Ethyl 2-[6-(2-methylphenyl)-2-(3-azabicyclo[3.3.0]oct-3-yl-amino)-7-oxopyrido[2,3-d]pyrimidin-8(7H)-yl]propanoate (Compound No. 21)
6-(2-Fluorophenyl)-2-(3-azabicyclo[3.3.0]oct-3-yl-amino)-8-(cyclopentyl)pyrido[2,3-d]pyrimidin-7(8h)-one (Compound No. 22)
6-(2-Fluorophenyl)-2-(3-azabicyclo[3.3.0]oct-3-yl-amino)-8-(cyclopropylmethyl)-pyrido[2,3-d]pyrimidin-7(8H)-one (Compound No. 23)
6-(2-Methylphenyl)-2-(3-azabicyclo[3.3.0]oct-3-yl-amino)-8-(2-chloroethyl)pyrido[2,3-d]pyrimidin-7(8H)-one (Compound No. 24)
6-(2-Chlorophenyl)-2-(3-azabicyclo[3.3.0]oct-3-yl-amino)-8-(2-ethoxyethyl)pyrido[2,3-d]pyrimidin-7(8H)-one (Compound No. 25)
2-[6-(2-Chlorophenyl)-2-(3-azabicyclo[3.3.0]oct-3-yl-amino)-7-oxopyrido[2,3-d]pyrimidin-8(7H)-yl]ethyl acetate (Compound No. 26)
6-(2-Chlorophenyl)-2-(3-azabicyclo[3.3.0]oct-3-yl-amino)-8-(2-methoxyethyl)pyrido[2,3-d]pyrimidin-7(8H)-one (Compound No. 27)
6-(2-Methylphenyl)-2-(3-azabicyclo[3.3.0]oct-3-yl-amino)-8-[2-(2-methoxyethoxy)ethyl]-pyrido[2,3-d]pyrimidin-7(8H)-one (Compound No. 28)
6-(2-Chlorophenyl)-2-(3-azabicyclo[3.3.0]oct-3-yl-amino)-8-[2-(2-methoxyethoxy)ethyl]pyrido[2,3-d]pyrimidin-7(8H)-one (Compound No. 29)
(2-{2-[2-(3-azabicyclo[3.3.0]oct-3-yl-amino)-6-(2-methylphenyl)-7-oxopyrido[2,3-d]pyrimidin-8(7H)-yl]ethyl}-1H-isoindole-1,3(2H)-dione (Compound No. 34)
Ethyl [2-(3-azabicyclo[3.3.0]oct-3-yl-amino)-6-(2-methylphenyl)-7-oxopyrido[2,3-d]pyrimidin-8(7H)-yl]acetate (Compound No. 44)

### Example 4

### Synthesis of tert-butyl 4-{[2-(3-azabicyclo[3.3.0]oct-3-yl-amino)-6-(2-methylphenyl)-7-oxopyrido[2,3-d]pyrimidin-8(7H)-yl]methyl}piperidine-1-carboxylate (Compound No. 32)

### Step a: 4-(2-Methylthio-7-oxo-6-(2-methylphenyl)-7H-pyrido[2,3d]-pyrimidin-8-ylmethyl)-piperidine-1-carboxylic acid tert-butyl ester

To the compound 6-(2-Methylphenyl)-2-methylthio-8H-pyrido[2,3-d]pyrimidin-7-one (0.500 g, 1.6 mmol) was added N-tert-butoxy carbonyl piperidine-4-methanol (0.750 g, 3.47 mmol), triphenylphosphine (0.91 g, 3.47 mmol) and evacuated for 30 minutes followed by the addition of dimethylformamide was added. To the reaction mixture was added diisopropylazadicarboxylate (0.71 g, 3.47 mmol) at 0° C. The reaction mixture was stirred at room temperature for 1 hour. The reaction mixture was poured into water and extracted with ethyl acetate, washed with water, dried over anhydrous sodium sulphate, and concentrated under reduced pressure. The residue thus obtained was purified by column chromatography using 20% ethyl acetate in hexane as eluent to furnish the title compound.

Yield=0.500 g.

The following illustrative intermediate was prepared analogously,
3-[2-Methylthio-7-oxo-6-(2-methylphenyl)-7H-pyrido[2,3d]pyrimidin-8-ylmethyl)-piperidine-1-carboxylic acid tert-butyl ester.

### Step b: 4-(2-Methylsulphonyl-7-oxo-6-(2-methylphenyl)-7H-pyrido[2,3d-pyrimidin-8-ylmethyl)-piperidine-1-carboxylic acid tert-butyl ester

The title compound was prepared following the procedure as described in Example 1, step e, by oxidizing the compound obtained from step a above.

### Step c: Tert-butyl 4-{[2-(3-azabicyclo [3.3.0]oct-3-yl-amino)-6-(2-methylphenyl)-7-oxopyrido[2,3-d]pyrimidin-8(7H)-yl]methyl}piperidine-1-carboxylate (Compound No. 32)

The title compound was prepared following the procedure as described in Example 1, step f. ¹H NMR (CDCl₃): δ 8.5 (1H, s, Ar—H), 7.48 (1H, s, Ar—H), 7.25-7.17 (4H, m, Ar—H), 4.36-4.34 (2H, m), 3.18 (2H, brs), 2.69 (6H, brs), 2.20 (3H, s, —CH₃), 1.75 (2H, s), 1.60-1.55 (6H, m), 1.4-1.26 (4H, m); Mass spectrum (m/z, +ve ion mode): 559 [M⁺+1].

Scheme III procedure

### Example 5

### Synthesis of 6-(2-methylphenyl)-2-(3-azabicyclo[3.3.0]oct-3-yl-amino)-8-[(1-acetylpiperidin-4-yl)methyl]-pyrido[2,3-d]pyrimidin-7(8H)-one (Compound No. 33)

### Step a: Hydrochloride salt of 2-methylthio-8-piperidin-3-yl-methyl-6-(2-methylphenyl)-8H-pyrido[2,3d]pyrimidin-7-one

To a solution of the compound 3-[2-methylthio-7-oxo-6-(2-methylphenyl)-7H-pyrido[2,3d]pyrimidin-8-ylmethyl)-piperidine-1-carboxylic acid tert-butyl ester (0.600 g) in dichloromethane was added etheral hydrochloric acid solution (2 ml) at 0° C. The reaction was stirred at room temperature for 2 hours. The reaction mixture was concentrated under reduced pressure. The residue thus obtained was purified by washing with diethylether to furnish the title compound, which was finally dried under reduced pressure.

### Step b: 8-(1-Scetyl-piperidine-3-ylmethyl)-2-methylthio)-6-(2-methylphenyl)-8H-pyrido[2,3d]pyrimidin-7-one

To the compound (0.350 mmol) obtained from step a above was added pyridine at 0° C. followed by the addition of acetic anhydride (2.0 ml) at 0° C. The reaction mixture was stirred at room temperature for 3 hours and subsequently diluted with water. The white solid thus obtained was filtered and dried under high vacuum to furnish the title compound.

### Step c: 8-(1-Acetyl-piperidine-3-ylmethyl)-2-methylsulphonyl)-6-(2-methylphenyl)-8H-pyrido[2,3d]pyrimidin-7-one

The title compound was prepared following the procedure as described Example 1, step e, by oxidizing the compound obtained from step b above.

### Step d: 6-(2-Methylphenyl)-2-(3-azabicyclo [3.3.0]oct-3-yl-amino)-8-[(1-acetylpiperidin-4-yl)methyl]-pyrido[2,3-d]pyrimidin-7(8H)-one (Compound No. 33)

The title compound was prepared following the procedure as described in Example 1, step f. Yield: 100 mg; ¹H NMR (CDCl₃): δ 8.53 (1H, s, Ar—H), 7.48 (1H, s, Ar—H), 7.22-7.17 (4H, m, Ar—H), 4.37-4.30 (3H, m), 3.2 (2H, brs), 2.91-2.89 (4H, m), 2.71-2.69 (2H, m), 2.22 (3H, s), 2.21-2.17 (2H, m), 1.97-1.74 (7H, m), 1.60-1.53 (6H, m); Mass spectrum (m/z, +ve ion mode): 501[M⁺+1].

### Scheme IV, Procedure

### Example 6

### Synthesis of N-{2-[2-(3-azabicyclo[3.3.0]oct-3-yl-amino)-6-(2-methylphenyl) -7-oxopyrido[2,3-d]pyrimidin-8(7H)-yl]ethyl}methanesulfonamide (Compound No. 38)

### Step a: 6-(2-Methylphenyl)-2-(3-azabicyclo [3.3.0]oct-3-yl-amino)-8-(2-aminoethyl)-pyrido[2,3-d]pyrimidin-7(8H)-one (Compound No. 35)

To a solution of the Compound No. 34 (0.90 g, 1.68 mmol) in ethanol (10 ml) was added aqueous solution of hydrazine (0.53 g, 16.8 mmol) and stirred the reaction mixture for 4 hours at room temperature. The solvent was evaporated under reduced pressure and the residue thus obtained was dissolved in ethanol. The mixture was concentrated under reduced pressure to furnish the title compound. Yield: 790 mg.

### Step b: N-{2-[2-(3-azabicyclo [3.3.0]oct-3-yl-amino)-6-(2-methylphenyl)-7-oxopyrido[2,3-d]pyrimidin-8(7H)-yl]ethyl}methanesulfonamide (Compound No. 38)

To a solution of the compound obtained from step a above (0.80 g, 0.19 mmol) in dichloromethane (10 ml) was added methanesulphonyl chloride (0.034 g, 0.29 mmol) and triethylamine (0.060 g, 0.59 mmol) at 0° C. The reaction mixture was stirred for 2 hours and subsequently diluted with dichloromethane. The mixture was extracted with sodium bicarbonate solution. The organic layer was collected, dried over anhydrous sodium sulphate and concentrated under reduced pressure. The residue thus obtained was purified by preparative column chromatography using 10% methanol in dichloromethane as eluent to furnish the title compound.

Yield: 23 mg; ¹H NMR (CDCl₃): δ 8.54 (s, 1H, Ar—H), 7.52 (s, 1H, Ar—H), 7.30-7.17 (m, 4H, Ar—H), 3.66 (brs, 11H, —NCH), 3.492 (m, 2H, —NCH₂), 3.40-3.34 (m, 5H, 2x-NCH₂ & —NCH), 2.99 (s, 3H, —SO₂CH₃), 2.54-2.53 (m, 2H, 2x-CH), 2,21 (s, 3H, Ar—CH₃), 1.71-1.54 (m, 6H, 3x-CH₂); Mass spectrum (m/z, +ve ion mode): 483 [M⁺+1].

The following illustrative compounds were prepared analogously,
N-{2-[2-(3-azabicyclo[3.3.0]oct-3-yl-amino)-6-(2-methylphenyl)-7-oxopyrido[2,3-d]pyrimidin-8(7H)-yl]ethyl}propane-1-sulfonamide (Compound No. 39)
N-{2-[2-(3-azabicyclo[3.3.0]oct-3-yl-amino)-6-(2-methylphenyl)-7-oxopyrido[2,3-d]pyrimidin-8(7H)-yl]ethyl}butane-1-sulfonamide (Compound No. 40)

### Example 7

### Synthesis of N-{2-[2-(3-azabicyclo[3.3.0]oct-3-yl-amino)-6-(2-methylphenyl) -7-oxopyrido[2,3-d]pyrimidin-8(7H)-yl]ethyl}acetamide (Compound No. 361

To a solution of the Compound No. 34 (0.80 g, 0.19 mmol) in dichloromethane (10 ml) was added acetyl chloride (0.02 g, 0.29 mmol) and triethylamine (0.058 g, 3.0 mmol) at 0° C. under nitrogen atmosphere. The reaction mixture was stirred for 4 hours. The reaction mixture was diluted with dichloromethane (20 ml) and extracted with sodium bicarbonate solution. The organic layer was separated, dried over anhydrous sodium sulphate and concentrated under reduced pressure. The residue thus obtained was purified by preparative column chromatography by using 10% methanol in dichloromethane as eluent to furnish the title compound. Yield: 13 mg; ¹H NMR (CDCl₃): δ 8.56 (s, 11H, Ar—H), 7.52 (s, 1H, Ar—H), 7.32-7.16 (m, 4H, Ar—H), 3.66-3.58 (m, 4H, —NCH₂+−NCH₂), 3.37-3.32 (m, 4H, 2x-NCH₂), 2.54-2.47 (m, 2H, 2x-CH), 2.21 (s, 3H, Ar—CH₃), 1.90 (s, 3H, —COCH₃), 1.72-1.26 (m, 6H, 3x-CH₂); Mass spectrum (m/z, +ve ion mode): 447 [M⁺+12].

The following illustrative compound was prepared analogously,
N-{2-[2-(3-azabicyclo[3.3.0]oct-3-yl-amino)-6-(2-methylphenyl)-7-oxopyrido[2,3-d]pyrimidin-8(7H)-yl]ethyl}propanamide (Compound No. 37)

### Scheme V, Procedure

### Example 8

### Synthesis of 6-(2-methylphenyl)-2-(3-azabicyclo[3.3.0]oct-3-yl-amino)-7-oxopyrido[2,3-d]pyrimidin-8(7H)-yl]acetic acid (Compound No. 79)

To a solution of the Compound No. 44 (0.3 g, 0.69 mmol) in methanol: tetrahydrofuran:water (1:3:1, 10 ml) was added lithium hydroxide (0.0668, 2.77 mmol) at 0° C. and the reaction mixture was stirred for 20 minutes. The mixture was allowed to warm up to room temperature and stirred for 4 hours. The solvents were removed under reduced pressure. The aqueous layer was extracted with diethyl ether. The organic layer was discarded and aqueous layer was collected, which was neutralized with hydrochloric acid (1 N) to maintain the pH at 2-3. The mixture was extracted with ethyl acetate and the organic layer was collected, dried over anhydrous sodium sulphate and concentrated under reduced pressure to furnish the title compound. Yield: 0.21 g; ¹H NMR (CDCl₃): δ 8.45 (1H, s, Ar—H), 7.57 (1H, s, Ar—H), 7.31-7.17 (4H, m, Ar—H), 5.30 (2H, s, —NCH₂COOH), 3.49-3.46 (2H, t, J=3.0 and 6 Hz, —NCH₂), 2.74 (brs, 2H, —NCH₂), 2.4-2.39 (d, 2H, J=3 Hz, 2x-CH), 2.23-2.18 (m, 5H, Ar—CH₃ & —CH₂), 1.44-1.19 (m, 4H, 2x-CH₂); Mass spectrum (m/z, +ve ion mode): 420 [M⁺+1].

### Example 9

### Synthesis of N-cyclopropyl-2-[2-(3-azabicyclo[3.3.0]oct-3-yl-amino)-6-(2-methylphenyl)-7-oxopyrido[2,3-d]pyrimidin-8(7H)-yl]acetamide (Compound No. 80)

To a solution of the Compound No. 79 (0.10 g, 0.24 mmol) in tetrahydrofuran (10 ml) at 0° C. was added N-methylmorpholine (0.074 g, 0.72 mmol), hydroxybenzotriazole (0.03 g, 0.24 mmol) and cyclopropylamine (0.014 g, 0.24 mmol). The reaction mixture was stirred at the same temperature for 20 minutes followed by the addition of 1-(3-dimethylaminopropyl)-3-ethylcarbodiimide hydrochloride (0.059 g, 0.3 mmol) and stirred the reaction mixture for 2 hours at room temperature. The solvent was evaporated under reduced pressure and the residue thus obtained was dissolved in ethyl acetate and extracted with water. The organic layer was separated, dried over anhydrous sodium sulphate and concentrated under educed pressure. The residue thus obtained was purified by column chromatography using 10% methanol in dichloromethane as eluent to furnish the title compound. Yield: 32 mg; ¹H NMR (CDCl₃): δ 8.53 (1H, s, Ar—H), 7.539 (1H, s, Ar—H), 7.37-7.36 (2H, d, J=4.5 Hz, Ar—H), 5.03 (1H, brs, —NH), 3.65 (1H, s, —NCH), 3.37-3.34 (3H, dd, J=9.0 Hz, —NCH₂+—NCH), 2.72-2.66 (3H, m, —NCH₂, —CH), 2.407 (1H, brs, —CH), 2.21 (3H, s, Ar—H), 1.69 (6H, m, 3x-CH₂), 0.75-0.71 (2H, m, —CH₂), 0.508 (2H, brs, —CH₂); Mass spectrum (m/z, +ve ion mode): 459 [M⁺+1].

The following illustrative compounds were prepared analogously,
6-(2-Methylphenyl)-2-(3-azabicyclo[3.3.0]oct-3-yl-amino)-7-oxopyrido[2,3-d]pyrimidin-8(7H)-yl-N-methoxyacetamide (Compound No. 46),
N-Cyclobutyl-2-[2-(3-azabicyclo[3.3.0]oct-3-yl-amino)-6-(2-methylphenyl)-7-oxopyrido[2,3-d]pyrimidin-8(7H)-yl]acetamide (Compound No. 47),
6-(2-Methylphenyl)-2-(3-azabicyclo[3.3.0]oct-3-yl-amino)-8-(2-oxo-2-pyrrolidin-1-ylethyl)pyrido[2,3-d]pyrimidin-7(8H)-one (Compound No. 48),
6-(2-Methylphenyl)-2-(3-azabicyclo[3.3.0]oct-3-yl-amino)-7-oxopyrido[2,3-d]pyrimidin-8(7H)-yl-N-isopropylacetamide (Compound No. 49).

### Example 10

### Synthesis of 6-(2-methylphenyl)-2-(3-azabicyclo[3.3.0]oct-3-yl-amino)-8-(2-hydroxyethyl)pyrido[2,3-d]pyrimidin-7(8H)-one (Compound No. 45)

To a solution of the Compound No. 44 (0.35 g, 0.80 mmol) in ethanol: tetrahydrofuran mixture (1:1, 30 ml) at 0° C. was added sodium borohydride (0.076 g, 2.0 mmol) and stirred the reaction mixture at room temperature overnight. To the reaction mixture was added ice-cold saturated ammonium chloride solution. Solvents were evaporated under reduced pressure and the aqueous layer was extracted with ethyl acetate. The organic layer was collected, dried over anhydrous sodium sulphate and concentrated under reduced pressure. The residue thus obtained was purified by column chromatography using 70-80% ethyl acetate in hexane to furnish the title compound.

Yield: 35 mg; ¹H NMR (CDCl₃): δ 8.51 (1H, s, Ar—H), 7.51 (1H, brs, Ar—H), 7.50-7.12 (4H, m, Ar—H), 5.329-5.327 (2H, d, J=6.0 Hz, —CH₂OH), 3.93-3.91 (2H, t, J=7.5 Hz, —CH₂OH), 3.38-3.30 (2H, m, —NCH₂), 2.73 (2H, brs, —NCH₂), 2.47 (brs, 2H, 2x-CH), 2.21 (s, 3H, Ar—CH₃), 1.72-1.55 (6H, m, 2x-CH₂); Mass spectrum (m/z, +ve ion mode): 406 [M⁺+1].

Scheme VI, procedure

### Example 11

### Synthesis of 6-(2-methylphenyl)-2-(3-azabicyclo[3.3.0]oct-3-yl-amino)-8-(2-methoxyethyl)-pyrido[2,3-d]pyrimidin-7(8H)-one (Compound No. 50)

### Step a: 8-(2-Hydroxyethyl)-6-(2-methylphenyl)-2-(methylthio)pyrido[2,3-d]pyrimidin-7(8H)-one

To a solution of the compound 6-(2-methylphenyl)-2-(methylthio)pyrido[2,3-d]pyrimidin-7(8H)-one (1.0 g, 3.53 mmol) in acetone (20 ml) was added ethyl bromide (1.32, 1.06 mmol), potassium carbonate (2.43 g, 17.6 mmol) and tetra-butyl ammonium iodide (0.65 g, 1.75 mmol). The reaction mixture was refluxed for overnight. Solid thus separated out was filtered off and the organic solvent was evaporated under reduced pressure. The residue thus obtained was purified by column chromatography using 50% ethyl acetate in hexane as eluent to furnish the title compound.

### Step b: 8-(2-Methoxyethyl)-6-(2-methylphenyl)-2-(methylthio)pyrido[2,3-d]pyrimidin-7(8H)-one

A suspension of sodium hydride (0.098 g, 4 mmol) in dry dimethylformamide (5.0 ml) at 0° C. under nitrogen atmosphere was added the compound obtained from step a above (0.32 g, 1 mmol). The reaction mixture was stirred at same temperature for 1 hour. To the resulting reaction mixture was added iodomethane (0.567 g, 4.0 mmol) and stirred for 2 hours. The mixture was diluted with ice-cold water and extracted with ethyl acetate. The organic layer was collected, dried over anhydrous sodium sulphate and concentrated under reduced pressure. The residue thus obtained was purified by column chromatography using 25% ethyl acetate in hexane to furnish the title compound.

### Step c: 8-(2-Methoxyethyl)-6-(2-methylphenyl)-2-(methylsulfonyl)pyrido[2,3-d]pyrimidin-7(8H)-one

The title compound was prepared following the procedure as described in Example 1, step e, by oxidizing the compound obtained from step b above.

### Step d: 6-(2-Methylphenyl)-2-(3-azabicyclo[3.3.0]oct-3-yl-amino)-8-(2-methoxyethyl)-pyrido[2,3-d]pyrimidin-7(8H)-one (Compound No. 50)

The title compound was prepared following the procedure as described in Example 1, step f. Yield 15 mg; ¹H NMR (CDCl₃): δ 8.52 (1H, s, Ar—H), 7.44 (1H, s, Ar—H), 7.24-7.18 (4H, m, Ar—H), 5.31-5.30 (1H, brs, —NH), 3.75 (2H, brs, —OCH₂), 3.64 (2H, brs, —NCH₂), 3.59 (s, 1H, —OCH₃), 3.40 (2H, brs, —NCH₂), 2.63 (2H, brs, —NCH₂), 2.50-2.34 (2H, m, 2x-CH), 2.22 (3H, s, Ar—CH₃), 1.72-1.33 (6H, m, 3x-CH₂); Mass spectrum (m/z, +ve ion mode): 420 [M⁺+1].

The following illustrative compound was prepared analogously,
6-(2-Methylphenyl)-2-(3-azabicyclo[3.3.0]oct-3-yl-amino)-8-(2-ethoxyethyl)pyrido[2,3-d]pyrimidin-7(8H)-one (Compound No. 51).

### Methodology

### p38 Inhibition Assays—Inhibition of Phosphorylation of EGF Receptor Peptide

This assay was carried out in the presence of 10 mM MgCl₂, 25 mM β-glycerophosphate, 10% glycerol and 100 mM HEPES buffer at pH 7.6. For a typical IC₅₀ determination, a stock solution was prepared containing all of the above components and activated p38 (5 nM). The stock solution was aliquoted into vials. A fixed volume of DMSO or inhibitor in DMSO (final concentration of DMSO in reaction was 5%) was introduced to each vial, mixed and incubated for 15 minutes at room temperature.

EGF receptor peptide, KRELVEPLTPSGEAPNQALLR, a phosphoryl acceptor in p38-catalysed kinase reaction (1), was added to each vial to a final concentration of 200 μM. The kinase reaction was initiated with ATP (100 μm) and the vials were incubated at 30C. After 30 minutes, the reactions were quenched with equal volume of 10% trifluoroacetic acid (TFA).

The phosphorylated peptide was quantified by HPLC analysis. Separation of the phosphorylated peptide from the unphosphorylated peptide was achieved on a reverse phase column (Deltapak, 5 μM, C18 100D, part no. 011795) with a binary gradient of water and acetonitrile, each containing 0.1% TFA. IC₅₀ (concentration of inhibitor yielding 50% inhibition) was determined by plotting the % activity remaining against inhibitor concentration.

Specific exemplary compounds were investigated and exhibited IC₅₀ for the p38 enzyme assay of from about 10 μM to about 60 nM, for example from about 800 nM to abut 60 nM, or from about 300 nM to about 60 nM, or from about 150 nM to about 60 nM, or from about 100 nM to about 60 nM (Compound No. 13 formed a precipitate).

Cell based Assay for TNF-α release

### Method of Isolation of Human Peripheral Blood Mononuclear Cells:

Human whole blood was collected in vacutainer tubes containing EDTA as an anti coagulant. A blood sample (7 ml) was carefully layered over 5 ml PMN Cell Isolation Medium (Robbins Scientific) in a 15 ml round bottom centrifuge tubes. The sample was centrifuged at 450-500×g for 30-35 minutes in a swing-out rotor at room temperature. After centrifugation the top band of cells were removed and washed 3 times with PBS w/o calcium or magnesium. The cells were centrifuged at 400×g for 10 minutes at room temperature. The cells were resuspended in Macrophage Serum Free Medium (Gibco BRL) at concentration of 2 million cells/ml.

### LPS Stimulation of Human PBMNC's:

PBM cells (0.1 ml; 2 million/ml) were co-incubated with 0.1 ml of compound (10-0.41 μM, final concentration) for 1 hour in flat bottom 96 well microtiter plate. Compounds were dissolved in DMSO initially and diluted in TCM for a final concentration of 0.1% DMSO. LPS (Cal biochem, 20 ng/ml, final concentration) was then added at volume of 0.010 ml. Cultures were incubated overnight at 37° C.). Supernatant were then removed and tested by ELISA for TNF-α release. Viability was analyzed using MTT. After 0.1 ml supernatant was collected, 0.1 ml of 0.25 mg/ml of MTT was added to remaining 0.1 ml of cells. The cells were incubated at 37° C. for 2-4 hours, then the O.D was measured at 490-650 nm.

The TNF-α levels released in the culture medium was quantitated by ELISA. Inhibitory potency was expressed as IC₅₀. Compounds 1, 20-23, 25, 40, 44, 47, 50-52, 59, 68, 69 and 71 were tested, and these compounds showed IC₅₀ for p38 inhibitory activity of from about 3 μM to about 140 nM, for example from about 950 nM to about 140 nM, or from about 630 nM to about 140 nM, or from about 375 nM to about 140 nM, or from about 200 nM to about 140 nM.

1. A compound having the structure of Formula I 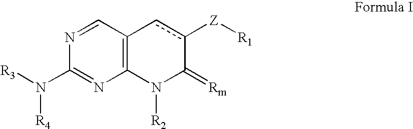
and its pharmaceutically acceptable salts, pharmaceutically acceptable solvates, esters, enantiomers diastereomers, N-oxides, polymorphs, metabolites;
wherein
R₁ is alkyl, alkenyl, alkynyl, cycloalkyl, aryl, aralkyl, heteroaryl, heterocyclyl, heteroarylalkyl, or heterocyclylalkyl;
when Rₘ is oxygen or sulphur
R₂ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y} or —NHC(═O)Rₓ; [symbol]
represents a single bond or a double bond;
R₃ is hydrogen, alkyl, alkenyl, alkynyl, cycloalkyl, aryl, heterocyclyl, heteroaryl, aralkyl, heteroarylalkyl or heterocyclylalkyl;
R₄ is 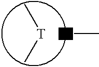
(wherein 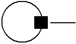
represents a cyclic ring containing 4-8 carbon atoms wherein 1-3 carbon atoms may optionally be replaced by heteroatoms selected from oxygen, —NH or sulphur; T is —(CH₂)ₙ—, —CH(Q)CH₂—, —CH₂CH(Q)CH₂—, —CH(Q)-, —CH₂—O—CH₂—, CH₂—NH—CH₂—, —CH₂—N(CH₃)—CH₂);
n is an integer selected from 0-3 (wherein when n is zero then T represents a direct bond);
R₅ is alkyl, alkenyl, alkynyl, cycloalkyl, —NRₚR_{q} (wherein Rₚ and R_{q} are independently selected from the group consisting of hydrogen, alkyl, alkenyl, alkynyl, cycloalkyl, aryl, aralkyl, heterocyclyl, heteroaryl, heterocyclylalkyl or heteroarylalkyl; Rₚ and R_{q} may also together join to form a heterocyclyl ring), aryl, aralkyl, heteroaryl, heterocyclyl, heterocyclylalkyl or heteroarylalkyl;
R₆ is alkyl, alkenyl, alkynyl, cycloalkyl, aryl, aralkyl, heteroarylalkyl or heterocyclylalkyl;
Z is a direct bond, oxygen, sulphur, —NH or —(CH₂)ₙ;
Rₓ and R_{y} are independently selected from the group consisting of hydrogen, alkyl, alkenyl, alkynyl, cycloalkyl, alkoxy, aryl, aralkyl, —SO₂R₅ (wherein R₅ is the same as defined above), heteroaryl, heterocyclyl, heteroarylalkyl or heterocyclylalkyl;
Q is alkyl, alkenyl, alkynyl, cycloalkyl, aryl, heteroaryl, heterocyclyl, aralkyl, heteroarylalkyl or heterocyclylalkyl.

2. A compound selected from the group consisting of:
6-(2-Chlorophenyl)-2-(3-azabicyclo[3.3.0]oct-3-yl-amino)-8-methylpyrido[2,3-d]pyrimidin-7(8H)-one (compound No. 1),
6-(2-Methylphenyl)-2-(3-azabicyclo[3.3.0]oct-3-yl-amino)-8-methylpyrido[2,3-d]pyrimidin-7(8H)-one (Compound No. 2),
6-(2-Fluorophenyl)-2-(3-azabicyclo[3.3.0]oct-3-yl-amino)-8-methylpyrido[2,3-d]pyrimidin-7(8H)-one (Compound No. 3),
6-(2,4-Difluorophenyl)-2-(3-azabicyclo[3.3.0]oct-3-yl-amino)-8-methylpyrido[2,3-d]pyrimidin-7(8H)-one (compound No. 4),
6-(3-Chlorophenyl)-2-(3-azabicyclo[3.3.0]oct-3-yl-amino)-8-methylpyrido[2,3-d]pyrimidin-7(8H)-one (Compound No. 5),
6-(4-Chlorophenyl)-2-(3-azabicyclo[3.3.0]oct-3-yl-amino)-8-methylpyrido[2,3-d]pyrimidin-7(8H)-one (Compound No. 6),
6-(4-Methylphenyl)-2-(3-azabicyclo[3.3.0]oct-3-yl-amino)-8-methylpyrido[2,3-d]pyrimidin-7(8H)-one (Compound No. 7),
6-(4-Fluorophenyl)-2-(3-azabicyclo[3.3.0]oct-3-yl-amino)-8-methylpyrido[2,3-d]pyrimidin-7(8H)-one (Compound No. 8),
6-(2-Methylphenyl)-2-(3-azabicyclo[3.3.0]oct-3-yl-amino)-8-methylpyrido[2,3-d]pyrimidin-7(8H)-one (Compound No. 9),
6-(2-methylphenyl)-2-(3-azabicyclo[3.3.0]oct-3-yl-amino)-8-(4-fluorobenzyl)-pyrido[2,3-d]pyrimidin-7(8H)-one (Compound No. 10),
6-(2-Methylphenyl)-2-(3-azabicyclo[3.3.0]oct-3-yl-amino)-8-(4-tert-butylbenzyl)-pyrido[2,3-d]pyrimidin-7(8H)-one (Compound No. 11),
6-(2-Methylphenyl)-2-(3-azabicyclo[3.3.0]oct-3-yl-amino)-8-(3-trifluoromethoxybenzyl)-pyrido[2,3-d]pyrimidin-7(8H)-one (Compound No. 12),
6-(2-Fluorophenyl)-2-(3-azabicyclo[3.3.0]oct-3-yl-amino)-8-(4-fluorobenzyl)-pyrido[2,3-d]pyrimidin-7(8H)-one (Compound No. 13),
6-(2-Fluorophenyl)-2-(3-azabicyclo[3.3.0]oct-3-yl-amino)-8-(3-chloro-2-fluorobenzyl)-pyrido[2,3-d]pyrimidin-7(8H)-one (Compound No. 14),
6-(2-Chlorophenyl)-2-(3-azabicyclo[3.3.0]oct-3-yl-amino)-8-methylpyrido[2,3-d]pyrimidin-7(8H)-one (Compound No. 15),
6-(2-Chlorophenyl)-2-(3-azabicyclo[3.3.0]oct-3-yl-amino)-8-(4-fluorobenzyl)-pyrido[2,3-d]pyrimidin-7(8H)-one (Compound No. 16),
6-(2-Chlorophenyl)-2-(3-azabicyclo[3.3.0]oct-3-yl-amino)-8-(4-tert-butylbenzyl)-pyrido[2,3-d]pyrimidin-7(8H)-one (Compound No. 17),
6-(2-Chlorophenyl)-2-(3-azabicyclo[3.3.0]oct-3-yl-amino)-8-(3-trifluoromethoxybenzyl)-pyrido[2,3-d]pyrimidin-7(8H)-one (Compound No. 18),
6-(2-Methylphenyl)-2-(3-azabicyclo[3.3.0]oct-3-yl-amino)-8-(cyclopropyl methyl)pyrido[2,3-d]pyrimidin-7(8H)-one (Compound No. 19),
Ethyl 2-[6-(2-fluorophenyl)-2-(3-azabicyclo[3.3.0]oct-3-yl-amino)-7-oxopyrido[2,3-d]pyrimidin-8(7H)-yl]propanoate (Compound No. 20),
Ethyl 2-[6-(2-methylphenyl)-2-(3-azabicyclo[3.3.0]oct-3-yl-amino)-7-oxopyrido[2,3-d]pyrimidin-8(7H)-yl]propanoate (Compound No. 21),
6-(2-Fluorophenyl)-2-(3-azabicyclo[3.3.0]oct-3-yl-amino)-8-(cyclopentyl)pyrido[2,3-d]pyrimidin-7(8H)-one (Compound No. 22),
6-(2-Fluorophenyl)-2-(3-azabicyclo[3.3.0]oct-3-yl-amino)-8-(cyclopropyl methyl)-pyrido[2,3-d]pyrimidin-7(8H)-one (Compound No. 23),
6-(2-Methylphenyl)-2-(3-azabicyclo[3.3.0]oct-3-yl-amino)-8-(2-chloroethyl)-pyrido[2,3-d]pyrimidin-7(8H)-one (Compound No. 24),
6-(2-Chlorophenyl)-2-(3-azabicyclo[3.3.0]oct-3-yl-amino)-8-(2-ethoxy ethyl)pyrido[2,3-d]pyrimidin-7(8H)-one (Compound No. 25),
2-[6-(2-Chlorophenyl)-2-(3-azabicyclo[3.3.0]oct-3-yl-amino)-7-oxopyrido[2,3-d]pyrimidin-8(7H)-yl]ethyl acetate (Compound No. 26),
6-(2-Chlorophenyl)-2-(3-azabicyclo[3.3.0]oct-3-yl-amino)-8-(2-methoxyethyl)pyrido[2,3-d]pyrimidin-7(8H)-one (Compound No. 27),
6-(2-methylphenyl)-2-(3-azabicyclo[3.3.0]oct-3-yl-amino)-8-[2-(2-methoxyethoxy)ethyl]-pyrido[2,3-d]pyrimidin-7(8H)-one (Compound No. 28),
6-(2-Chlorophenyl)-2-(3-azabicyclo[3.3.0]oct-3-yl-amino)-8-[2-(2-methoxyethoxy)ethyl]pyrido[2,3-d]pyrimidin-7(8H)-one (Compound No. 29),
6-(2-Methylphenyl)-2-(3-azabicyclo[3.3.0]oct-3-yl-amino)-pyrido[2,3-d]pyrimidin-7(8H)-one (Compound No. 30),
6-(2-Fluorophenyl)-2-(3-azabicyclo[3.3.0]oct-3-yl-amino)pyrido[2,3-d]pyrimidin-7(8H)-one (Compound No. 31),
tert-butyl 4-{[2-(3-azabicyclo[3.3.0]oct-3-yl-amino)-6-(2-methylphenyl)-7-oxopyrido[2,3-d]pyrimidin-8(7H)-yl]methyl}piperidine-1-carboxylate (Compound No. 32),
6-(2-Methylphenyl)-2-(3-azabicyclo[3.3.0]oct-3-yl-amino)-8-[(1-acetylpiperidin-4-yl)methyl]-pyrido[2,3-d]pyrimidin-7(8H)-one (Compound No. 33),
2-{2-[2-(3-azabicyclo[3.3.0]oct-3-yl-amino)6-(2-methylphenyl)-7-oxopyrido[2,3-d]pyrimidin-8(7H)-yl]ethyl}-1H-isoindole-1,3(2H)-dione (Compound No. 34),
6-(2-Methylphenyl)-2-(3-azabicyclo[3.3.0]oct-3-yl-amino)-8-(2-aminoethyl)-pyrido[2,3-d]pyrimidin-7(8H)-one (Compound No. 35),
N-{2-[2-(3-azabicyclo[3.3.0]oct-3-yl-amino)-6-(2-methylphenyl)-7-oxopyrido[2,3-d]pyrimidin-8(7H)-yl]ethyl}acetamide (Compound No. 36),
N-{2-[2-(3-azabicyclo[3.3.0]oct-3-yl-amino)-6-(2-methylphenyl)-7-oxopyrido[2,3-d]pyrimidin-8(7H)-yl]ethyl}propanamide (Compound No. 37),
N-{2-[2-(3-azabicyclo[3.3.0]oct-3-yl-amino)-6-(2-methylphenyl)-7-oxopyrido[2,3-d]pyrimidin-8(7H)-yl]ethyl}methanesulfonamide (Compound No. 38),
N-{2-[2-(3-azabicyclo[3.3.0]oct-3-yl-amino)-6-(2-methylphenyl)-7-oxopyrido[2,3-d]pyrimidin-8(7H)-yl]ethyl}propane-1-sulfonamide (Compound No. 39),
N-{2-[2-(3-azabicyclo[3.3.0]oct-3-yl-amino)-6-(2-methylphenyl)-7-oxopyrido[2,3-d]pyrimidin-8(7H)-yl]ethyl}butane-1-sulfonamide (Compound No. 40),
6-(2-Methylphenyl)-2-(7-azabicyclo[4.3.0]non-7-yl-amino)-8-(cyclopropyl)-pyrido[2,3-d]pyrimidin-7(8H)-one (Compound No. 41),
6-(2-Methylphenyl)-2-(7-azabicyclo[4.3.0]non-7-yl-amino)-8-methyl-pyrido[2,3-d]pyrimidin-7(8H)-one (Compound No. 42),
6-(2-Chlorophenyl)-2-(7-azabicyclo[4.3.0]non-7-yl-amino)-8-methyl-pyrido[2,3-d]pyrimidin-7(8H)-one (Compound No. 43),
Ethyl[2-(3-azabicyclo[3.3.0]oct-3-yl-amino)-6-(2-methylphenyl)-7-oxopyrido[2,3-d]pyrimidin-8(7H)-yl]acetate (Compound No. 44),
6-(2-Methylphenyl)-2-(3-azabicyclo[3.3.0]oct-3-yl-amino)-8-(2-hydroxyethyl)pyrido[2,3-d]pyrimidin-7(8H)-one (Compound No. 45),
6-(2-Methylphenyl)-2-(3-azabicyclo[3.3.0]oct-3-yl-amino)-7-oxopyrido[2,3-d]pyrimidin-8(7H)-yl-N-methoxyacetamide (Compound No. 46),
N-Cyclobutyl-2-[2-(3-azabicyclo[3.3.0]oct-3-yl-amino)-6-(2-methylphenyl)-7-oxopyrido[2,3-d]pyrimidin-8(7H)-yl]acetamide (Compound No. 47),
6-(2-Methylphenyl)-2-(3-azabicyclo[3.3.0]oct-3-yl-amino)-8-(2-oxo-2-pyrrolidin-1-ylethyl)pyrido[2,3-d]pyrimidin-7(8H)-one (Compound No. 48),
6-(2-Methylphenyl)-2-(3-azabicyclo[3.3.0]oct-3-yl-amino)-7-oxopyrido[2,3-d]pyrimidin-8(7H)-yl-N-isopropylacetamide (Compound No. 49),
6-(2-Methylphenyl)-2-(3-azabicyclo[3.3.0]oct-3-yl-amino)-8-(2-methoxyethyl)-pyrido[2,3-d]pyrimidin-7(8H)-one (Compound No. 50),
6-(2-Methylphenyl)-2-(3-azabicyclo[3.3.0]oct-3-yl-amino)-8-(2-ethoxyethyl)pyrido[2,3-d]pyrimidin-7(8H)-one (Compound No. 51),
6-(2-Chlorophenyl)-2-(3-azabicyclo[3.3.0]oct-3-yl-amino)-8-cyclopropyl-pyrido[2,3-d]pyrimidin-7(8H)-one (Compound No. 52),
6-(2-Chlorophenyl)-2-(3-azabicyclo[3.3.0]oct-3-yl-amino)-7-oxopyrido[2,3-d]pyrimidin-8(7H)-yl acetonitrile (Compound No. 53),
6-(1-Naphthyl)-2-(3-azabicyclo[3.3.0]oct-3-yl-amino)-8-cyclopropyl-pyrido[2,3-d]pyrimidin-7(8H)-one (Compound No. 54),
6-(2-Chlorophenoxy)-2-(3-azabicyclo[3.3.0]oct-3-yl-amino)-8-cyclopropyl-pyrido[2,3-d]pyrimidin-7(8H)-one (Compound No. 55),
6-(3-Methylphenyl)-2-(3-azabicyclo[3.3.0]oct-3-yl-amino)-8-methyl-pyrido[2,3-d]pyrimidin-7(8H)-one (Compound No. 56),
6-(2-Methylphenoxy)-2-(3-azabicyclo[3.3.0]oct-3-yl-amino)-8-cyclopropyl-pyrido[2,3-d]pyrimidin-7(8H)-one (Compound No. 57),
6-(2-Methylphenoxy)-2-(3-azabicyclo[3.3.0]oct-3-yl-amino)-8-methylpyrido[2,3-d]pyrimidin-7(8H)-one (Compound No. 58),
6-(2-Fluorophenoxy)-2-(3-azabicyclo[3.3.0]oct-3-yl-amino)-8-cyclopropyl-pyrido[2,3-d]pyrimidin-7(8H)-one (Compound No. 59),
6-(4-Chlorophenyl)-2-(3-azabicyclo[3.3.0]oct-3-yl-amino)-8-cyclopropylpyrido[2,3-d]pyrimidin-7(8H)-one (Compound No. 60),
6-(4-Chlorophenoxy)-2-(3-azabicyclo[3.3.0]oct-3-yl-amino)-8-methylpyrido[2,3-d]pyrimidin-7(8H)-one (Compound No. 61),
6-Methoxy-2-(3-azabicyclo[3.3.0]oct-3-yl-amino)-8-methylpyrido[2,3-d]pyrimidin-7(8H)-one (Compound No. 62),
6-(4-Isopropylphenyl)-2-(3-azabicyclo[3.3.0]oct-3-yl-amino)-8-methylpyrido[2,3-d]pyrimidin-7(8H)-one (Compound No. 63),
6-(2-Fluorophenyl)-2-(3-azabicyclo[3.3.0]oct-3-yl-amino)-8-(cyclopropyl)pyrido[2,3-d]pyrimidin-7(8H)-one (Compound No. 64),
6-(2,4-Dichlorophenyl)-2-(3-azabicyclo[3.3.0]oct-3-yl-amino)-8-methylpyrido[2,3-d]pyrimidin-7(8H)-one (Compound No. 65),
6-(4-Nitrophenyl)-2-(3-azabicyclo[3.3.0]oct-3-yl-amino)-8-methyl-pyrido[2,3-d]pyrimidin-7(8H)-one (Compound No. 66),
6-(3-Methoxyphenyl)-2-(3-azabicyclo[3.3.0]oct-3-yl-amino)-8-methylpyrido[2,3-d]pyrimidin-7(8H)-one (Compound No. 67),
6-(2,6-Dichlorophenyl)-2-(3-azabicyclo[3.3.0]oct-3-yl-amino)-8-methylpyrido[2,3-d]pyrimidin-7(8H)-one (Compound No. 68),
6-(2-Nitrophenyl)-2-(3-azabicyclo[3.3.0]oct-3-yl-amino)-8-methyl-pyrido[2,3-d]pyrimidin-7(8H)-one (Compound No. 69),
6-(3-Fluorophenyl)-2-(3-azabicyclo[3.3.0]oct-3-yl-amino)-8-methylpyrido[2,3-d]pyrimidin-7(8H)-one (Compound No. 70),
6-(2-Chloro-4-fluorophenyl)-2-(3-azabicyclo[3.3.0]oct-3-yl-amino)-8-methylpyrido[2,3-d]pyrimidin-7(8H)-one (Compound No. 70),
6-(4-Nitrophenoxy)-2-(3-azabicyclo[3.3.0]oct-3-yl-amino)-8-methyl-pyrido[2,3-d]pyrimidin-7(8H)-one (Compound No. 72),
6-(2-Chlorophenoxy)-2-(3-azabicyclo[3.3.0]oct-3-yl-amino)-8-methylpyrido[2,3-d]pyrimidin-7(8H)-one (Compound No. 73),
6-(2-Fluorophenoxy)-2-(3-azabicyclo[3.3.0]oct-3-yl-amino)-8-methylpyrido[2,3-d]pyrimidin-7(8H)-one (Compound No. 74),
6-(4-Chloro-2-fluorophenoxy)-2-(3-azabicyclo[3.3.0]oct-3-yl-amino)-8-methylpyrido[2,3-d]pyrimidin-7(8H)-one (Compound No. 75),
6-(4-Chloro-2-fluorophenoxy)-2-(3-azabicyclo[3.3.0]oct-3-yl-amino)-8-cyclopropyl-pyrido[2,3-d]pyrimidin-7(8H)-one (Compound No. 76),
6-(3-Methoxyphenoxy)-2-(3-azabicyclo[3.3.0]oct-3-yl-amino)-8-(cyclopropyl)-pyrido[2,3-d]pyrimidin-7(8H)-one (Compound No. 77),
6-(3-Methylphenoxy)-2-(3-azabicyclo[3.3.0]oct-3-yl-amino)-8-(cyclopropyl)-pyrido[2,3-d]pyrimidin-7(8H)-one (Compound No. 78),
6-(2-Methylphenyl)-2-(3-azabicyclo[3.3.0]oct-3-yl-amino)-7-oxopyrido[2,3-d]pyrimidin-8(7H)-yl]acetic acid (Compound No. 79),
N-Cyclopropyl-2-[2-(3-azabicyclo[3.3.0]oct-3-yl-amino)-6-(2-methylphenyl)-7-oxopyrido[2,3-d]pyrimidin-8(7H)-yl]acetamide (Compound No. 80).

3. A method for treating mammals suffering from inflammation and associated pathologies, the method comprising administration of a therapeutically effective amount of a compound of claim 1.

4. A method for treating mammals suffering from sepsis, rheumatoid arthritis, inflammatory bowel disease, type-1 diabetes, asthma, chronic obstructive pulmonary disorder, organ transplant rejection, acute coronary syndrome and psoriasis, the method comprising administration of a therapeutically effective amount of a compound of claim 1.

5. A pharmaceutical composition for the treatment of inflammation and associated pathologies, the composition comprising a compound of claim 1, and a pharmaceutically acceptable carrier.

6. A pharmaceutical composition for treating mammals suffering from sepsis, rheumatoid arthritis, inflammatory bowel disease, type-1 diabetes, asthma, chronic obstructive pulmonary disorder, organ transplant rejection, acute coronary syndrome and psoriasis, the composition comprising a compound of claim 1, and pharmaceutically acceptable carrier.

7. A method for treating mammals suffering from inflammation and associated pathologies, the method comprising administration of a composition of claim 5.

8. A method according to claim 3, wherein the disease or disorder is mediated through p38 MAP Kinase.

9. A method of making a compound of Formula XI, or pharmaceutically acceptable salts or esters thereof, wherein the reaction comprises
a. reacting a compound of Formula II 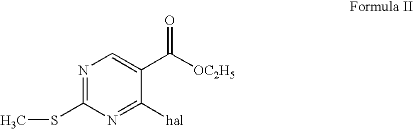
with a compound of Formula III
NH₂Rd Formula III
to give a compound of Formula IV; 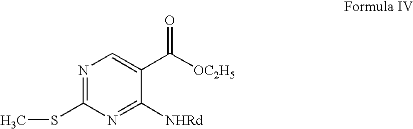
b. reducing a compound of Formula IV to give a compound of Formula V; 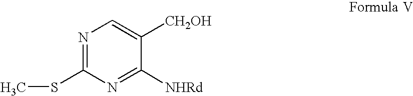
c. oxidizing a compound of Formula V to give a compound of Formula VI; 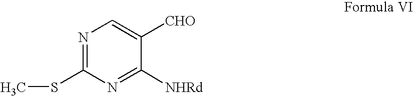
d. reacting a compound of Formula VI with an ester of Formula VII 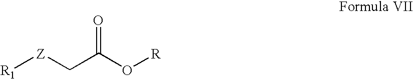
to give a compound of Formula VIII; 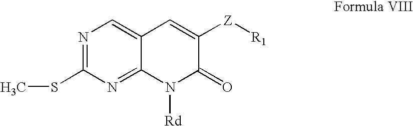
e. oxidizing a compound of Formula VII to give a compound of Formula IX; and 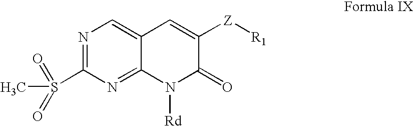
f. reacting a compound of Formula IX with a compound of Formula X 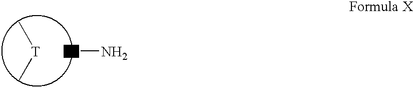
to give a compound of Formula XI, 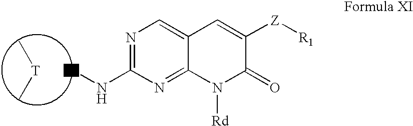
wherein
hal is halogen (Cl, Br or I);
R_{d} is hydrogen, optionally substituted alkyl, cycloalkyl, aralkyl or aryl;
R′ is alkyl;
R₁ is alkyl, alkenyl, alkynyl, cycloalkyl, aryl, aralkyl, heteroaryl, heterocyclyl, heteroarylalkyl, or heterocyclylalkyl;
Z is a direct bond, oxygen, sulphur, —NH or —(CH₂)ₙ;
n is an integer selected from 0-3 (wherein when n is zero then T represents a direct bond);
T is —(CH₂)ₙ—, —CH(Q)CH₂—, —CH₂CH(Q)CH₂—, —CH(Q)-, —CH₂—O—CH₂—, —CH₂—NH—CH₂—, —CH₂—N(CH₃)—CH₂);

10. A method of making a compound of Formula XVI, or pharmaceutically acceptable salts or esters thereof, wherein the reaction comprises
a. reacting a compound of Formula XII 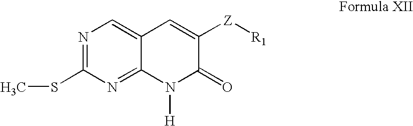
with a compound of Formula XIII
R₂ₐ-D Formula XIII
to give a compound of Formula XIV; 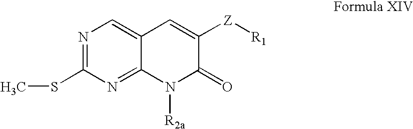
b. oxidizing a compound of Formula XIV to give a compound of Formula XV; and 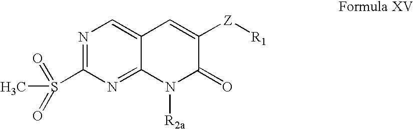
c. reacting a compound of Formula XV with a compound of Formula X 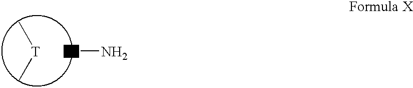
to give a compound of Formula XVI, 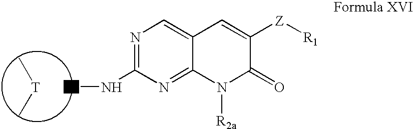
wherein
R₁ is alkyl, alkenyl, alkynyl, cycloalkyl, aryl, aralkyl, heteroaryl, heterocyclyl, heteroarylalkyl, or heterocyclylalkyl;
Z is a direct bond, oxygen, sulphur, —NH or —(CH₂)ₙ;
n is an integer selected from 0-3 (wherein when n is zero then T represents a direct bond);
T is —(CH₂)ₙ—, —CH(Q)CH₂—, —CH₂CH(Q)CH₂—, —CH(Q)-, —CH₂—O—CH₂—, —CH₂—NH—CH₂—, —CH₂—N(CH₃)—CH₂;
Q is alkyl, alkenyl, alkynyl, cycloalkyl, aryl, heteroaryl, heterocyclyl, aralkyl, heteroarylalkyl or heterocyclylalkyl.
R₂ₐ is hydrogen, alkyl, alkenyl, alkynyl, cycloalkyl, aryl, aralkyl, heteroaryl, heterocyclyl, heterocyclylalkyl or heteroarylalkyl;
D is hal or —OH;

11. (canceled)

12. (canceled)

13. (canceled)

14. (canceled)

15. (canceled)

16. (canceled)
